(19) United States
(12) Patent Application Publication
(10) Pub. No.: US 2004/0143863 A1
(43) Pub. Date: Jul. 22, 2004

(54) Hematopoietic stem cell niche cells

(76) Inventors: Linheng Li, Leawood, KS (US); Jiwang Zhang, Overland Park, KS (US)

Correspondence Address: POLSINELLI SHALTON & WELTE, P.C., 700 W. 47TH STREET, SUITE 1000, KANSAS CITY, MO, 64112-1802, US

(21) Appl. No.: 10/641,319

(22) Filed: Aug. 14, 2003

Related U.S. Application Data
(60) Provisional application No. 60/439,198, filed on Jan. 10, 2003.

(51) Int. Cl. A01K 67/027; C12N 15/63

(52) U.S. Cl. 800/18; 435/320.1

(57) ABSTRACT

The present invention relates to an isolated population of osteoblastic cells, which are characterized by cell surface markers N-cad⁺ and CD45⁻, with such osteoblastic cells used in vitro to promote and support growth of HSCs. Additionally, the present invention relates to vectors, which include a Bmpr1a nucleic acid sequence, recombination sites, and a plasmid, wherein the vectors can be used to promote an increase in the HSC population in vivo.

### FIELD OF INVENTION

The present invention relates to isolated osteoblast cells, which can be used as niche cells to support hematopoietic stem cells (HSC) in vitro. The present invention further relates to various compositions and methods for promoting increased HSC in vivo.

### BACKGROUND OF INVENTION

HSCs are a subset of bone marrow cells that possess the properties of both self-renewal and multilineage potential. In the past few decades, remarkable progress has been made in the identification of cytokines or stromal cells that promote HSC proliferation and differentiation. Thus far, however, the relative inability to expand HSCs in vitro has greatly hindered mechanistic studies of stem cell properties and imposed limitations on the use of these cells in transplantation. This is largely due to lack of adequate in vivo information regarding where HSCs reside, what the components of their microenvironment (niche) are, and which molecules are involved in the maintenance of HSCs. As such, identification of niches, and the components of niches, will allow for the isolation and purification of the niche cells which support HSCs. The niche cells have a potential to be used as feeder cells to expand stem cells in vitro just as mouse fibroblast cells are used to expand embryonic stem cells. In addition, in vitro co-culture of HSCs will facilitate further dissection of molecular control of stem cell self-renewal in a relative high-throughput way. Hematopoietic stem cells have been identified for more than 40 years, but, expanding HSCs in vitro is still challenging. Identification of cellular constitution of the niche that supports HSCs in vivo will open the avenue of maintaining and expanding HSCs in vitro.

Bone marrow stromal cells are derived from mesenchymal stem cells, with the bone marrow stromal cells, including fibroblasts, adipocytes, endothelial cells, and osteoblasts. Past studies regarding the roles of stromal cells in supporting HSCs have mainly been based on in vitro culture, and each of these stromal cells has been suggested to be capable of supporting hematopoietic stem/progenitor cells in vitro; however, this has not been confirmed in vivo. Additionally, more specific stromal cells have not been identified which can be used to support HSC in vitro, in particular, isolated sub-populations have not been identified.

Present technologies using either a combination of cytokines or stromal cells have several problems. Use of a combination of cytokines to culture HSCs can induce stem cells to proliferate but, invariably, induce stem cells to undergo concomitant differentiation. Different types of bone marrow derived stromal cells have been reported to be able to maintain stem cells in vitro in varying degrees, but none of this has been confirmed in vivo.

Several reports have shown that osteoblasts can support HSCs in vitro (Taichman et al., Hematology, 2000), or osteoblasts can increase the engraftment rate when co-transplanted with HSCs (El-Badri et al., Exp Hematology 1998), Gregory et al. also show that osteoblasts can support HSCs in vivo (ASH meeting, December 2002). But, none of these works has defined a particular cell lineage with cell surface markers. It is desired to identify the particular cell sub-population of the osteoblasts which support HSCs in vivo. Thus, it is desired to have a population of cells which can be identified, isolated, and used in vitro to promote and support growth of HSCs.

It is desired to have a method for increasing HSC population in vivo. In particular, it is desired to expand HSC in vivo in bone. It is also desired to have model organisms which can be used to analyze HSC in vivo. Tools which are used to form these organisms are also desired. Finally, it is desired to have cells which support HSC in vitro.

### SUMMARY OF INVENTION

The present invention relates to an isolated population of osteoblastic cells, which support HSCs in vitro. The osteoblast cells form niches in vivo that support HSC. In particular, the present invention relates to an isolated population of osteoblastic cells, which are characterized by cell surface markers N-cad⁺ and CD45⁻. The present invention also relates to methods for isolating the osteoblast population, as well as methods for supporting HSCs in vitro. The osteoblasts can be used to form feeder layers for supporting HSC.

Additionally, the present invention relates to vectors, which include a Bmpr1a nucleic acid sequence, recombination sites, and a plasmid. The vectors are used to produce knockout organisms. Antibodies and Fab fragments to the Bmpr1a polypeptide are also contemplated. The vectors and antibodies can be used to promote an increase in the HSC population in vivo.

The isolated population of N-cad⁺ CD45⁻ osteoblast cells are niche cells that support HSC. An in vitro population of cells can be formed that has N-cad⁺ CD45⁻ osteoblast cells and HSC. The HSC will include Lin⁻Sca-1⁺c-Kit⁺CD45⁺N-cad⁺ HSC. The niche cells are isolated by mixing a population of stem cells with labeled cell surface markers. The cell surface markers are CD45 and N-cadherin, with the labeled cells passed through a FACS sorter to separate N-cad⁺CD45⁻ cells from the remaining population.

The vector, preferably is an inducible Cre/Flp recombinase expression vector, with the recombination sites being LoxP sites. The vector can include sequences homologous to the Bmpr1a nucleic acid sequence so that when recombination occurs, Bmpr1a is inhibited. As such, the method is initiated by forming a vector which includes the Bmpr1a sequence. The vector is contacted with embryonic stem cells. A second vector which expresses recombinase is also contacted with a separate group of embryonic stem cells. Both sets of transfected embryogenic stem cells are transplanted into separate females. The embryos are allowed to gestate, and the resultant adults are then crossed to form selected host progeny that contains the recombination site and recombinase. Thus, a host organism can be transfected with a homologous Bmpr1a recombination sequence and a regulatory element. This will include an inducible Cre/lox system, whereby Bmpr1a is flanked by LoxP sites. In particular, a mouse can be transfected with the vector. Specifically, an Mx1-Cre⁺ Bmpr1a^{fx/fx} mouse can be formed. A knockout mouse results, wherein the Bmpr1a gene has been substantially eliminated.

The method for increasing HSC in vivo includes forming a Bmpr1a recombination vector; transfecting a host with the vector; and, inducing recombination in the host, wherein the Bmpr1a gene is knocked-out. Preferably, the vector is initially delivered to embryonic cells. Delivery of the sequence can be done via transfection, electroporation, or microinjection. As such, cassettes can be used in place of vectors.

Antibodies to the Bmpr1a polypeptide can be made. Also, Fab fragments to the antibody can be made. An antibody to the Bmpr1a polypeptide that inhibits such polypeptide will increase HSC in vivo.

### BRIEF DESCRIPTION OF THE DRAWINGS

The application file contains at least one photograph executed in color. Copies of this patent application publication with color photographs will be provided by the Office upon request and payment of the necessary fee.

FIG. 1 illustrates expression patterns of Bmpr1a & b bone and bone marrow cells and detection of conditional deletion of the Bmpr1a gene;

FIG. 1a is a schematic illustration of bone and the bone marrow structures;

FIG. 1b illustrates expression of Bmpr1a detected in osteoblastic cells and bone marrow cells;

FIG. 1c illustrates detection of Bmpr1a gene expression in hematopoietic stem and lineage cells using RT-PCR assay;

FIG. 1d illustrates detection of Bmpr1b gene expression in hematopoietic stem and lineage cells using RT-PCR assay;

FIG. 2a shows the results of GFP targeting resulting from Polyl:C-induced MX1-Cre recombination in Z/EG reporter mice, OB: osteoblasts, TB: trabecular bone;

FIG. 2b is a schematic illustration of the Bmpr1a gene structure in which a pair of LoxP sequences had been integrated to flank Exon2, recombination results are also illustrated;

FIG. 2c illustrates electrophoretic gel picture of PCR analysis on the genomic DNA derived from hematopoietic lineages of Bmpr1a mutant mice 3-months post Poly I:C injection (three time), the 2500 bp DNA band indicates the pre-excision state and the 180 bp DNA band indicates the post-excision state;

FIG. 3a illustrates a FACSFlow assay on an HSC population, the percentage shown in the gate is the ratio of HSCs in mononuclear BM cells, the panels relate to a specific mouse genotype;

FIG. 3b shows a comparison of absolute number of HSCs per femur in wild type (Wt) and the Bmpr1a mutant mice;

FIG. 3c shows a comparison of the number of LT-HSCs (BrdU-negative Lin⁻c-Kit⁺Sca-1⁺) and ST-HSCs (BrdU-positive Lin⁻c-Kit⁺Sca-1⁺) in the Bmpr1a mutant and Wt control mice;

FIG. 4a shows a competitive repopulation unit (CRU) assay to compare the number of functional HSCs per femur between the Bmpr1a mutant and Wt control mice;

FIG. 4b is a schematic drawing showing transplantation of HSCs derived from Wt mice into Wt and the Bmpr1a mutant recipient mice;

FIG. 4c shows an analysis of percentage of HSCs (mixture of LT- and ST-HSCs) in recipient (Bmpr1a mutant and Wt control) mice;

FIG. 4d is a schematic drawing showing transplantation of BM cells from Wt and the Bmpr1a mutant mice into Wt recipient mice;

FIG. 4e is an analysis of percentage of HSCs (mixture of LT- and ST-HSCs) in Wt Ly5.1 recipient mice;

FIG. 5. illustrates x-ray and histological analyses of bone structure, comparison of the HSC number among early, late, and combined early+late induced Bmpr1a mutant and Wt control mice, analysis of triple-genotype mouse model, and count of N-cadherin⁺ osteoblastic cell number, comparison of the location of BrdU-LTR (long-term retaining) cells between the Bmpr1a mutant and Wt control bone/BM sections, distribution of the spindle-shaped N-cadherin⁺ osteoblastic cells;

FIG. 5a is an x-ray image of the bone structures of both front and hind legs in the late-induced mutant mice, the red arrow indicates ectopically formed trabecular bone-like area (TBLA) and the dotted line indicates where cross-sections were taken;

FIGS. 5b-5c illustrate H&E staining of cross sections of femur where the TBLA was formed in the Bmpr1a mutant mice, and the corresponding long bone portion of Wt control mice;

FIGS. 5d-5f shows x-ray images of femur and tibia from early, late and combined early+late induced Bmpr1a mutant, and Wt control mice, the red arrows indicate TBLAs;

FIG. 5g shows a comparison of the absolute number of HSCs (mixture of LT- and ST-HSCs) per femur in early, late, and the combined early+late induced Bmpr1a mutant to Wt control mice.

FIG. 6a illustrates inactivation of Bmpr1a in the surface of ectopically formed TBLA by analyzing the triple-genotype mice, GFP expression represents successful Cre-mediated gene deletion, DAPI was used as a counterstaining;

FIGS. 6b-6d illustrate detection of BrdU-LTR cells in the regions of TBA, TBLA, and long bone, the blue arrow indicates matrix-forming osteoblasts, and the black arrow indicates spindle shaped N-cadherin⁺ osteoblastic cells;

FIGS. 6e-6g illustrates the distribution of spindle-shaped N-cadherin⁺ osteoblastic cells in the regions of TBA, TBLA, and long bone;

FIG. 6h displays a count of the number of the spindle-shaped N-cadherin⁺ osteoblastic cells in Wt control and the Bmpr1a mutant mice.

FIG. 6i illustrates a summary of distribution of three types of cell populations;

FIG. 7 shows the results of co-staining of BrdU-LTR cells with HSC markers Sca-1 and c-Kit, hematopoietic specific marker CD45, adherens junctions related molecules N-cadherin and β-catenin, DAPI was used to indicate nuclei. Yellow arrow indicates spindle shaped osteoblastic cells;

FIGS. 7a-7e show co-staining of BrdU-LTR cells with CD45 (a) Sca-1 (b) (frozen section), c-Kit (c) β-catenin (d) and N-cadherin (e) labels;

FIG. 7f shows an enlargement of the area of attachment of the BrdU-LTR cells to N-cadherin⁺ osteoblastic cells (e);

FIG. 8a are schematic models describing how the BMP signal regulates proliferation or differentiation of the osteoblastic progenitor;

FIG. 8b is a schematic model to illustrate the niche for hematopoietic stem cells;

FIG. 9 illustrates an analysis of the percentage of hematopoietic progenitors and mature blood cells in the Bmpr1a mutant and littermate control mice, different samples were stained with cell surface markers;

FIG. 9a shows the percentage results in peripheral blood in mutant and control mice;

FIG. 9b shows the percentage results in thymus T lineages in mutant and control mice;

FIG. 9c shows the percentage results in bone marrow myeloid lineages in mutant and control mice;

FIG. 9d shows the percentage results in thymus DN lineages in mutant and control mice;

FIG. 9e shows the percentage results in bone marrow B lineages in mutant and control mice; and,

FIG. 9f shows the percentage results in spleen B and T lineages in mutant and control mice.

FIG. 10 illustrates an analysis of common lymphoid progenitors (CLP) and common myeloid progenitors (CMP) in Bmpr1a mutant and Wt control mice using a flow cytometry assay, with the percentage of each population shown in a gate;

FIG. 10a relates to CLP and CMP in wild type mice;

FIG. 10b relates to CLP and CMP in the Bmpr1a mutant mice;

FIG. 11 illustrates a colony forming unit (CFU) culture assays on bone marrow cells derived from Bmpr1a mutant and Wt control mice;

FIG. 12a illustrates the histomorphometry of bone sections in wt and mutant Bmpr1a mice;

FIGS. 12b illustrates the bone forming rate in wt and mutant Bmpr1a mice;

FIG. 12c relates to measurement of trabecular bone volume in wt and mutant Bmpr1a mice;

FIG. 12d relates to the osteoblast number in wt and mutant Bmpr1a mice; and,

FIG. 13 shows the analysis of N-cadherin-low HSCs using a flow cytometry assay.

### DETAILED DESCRIPTION OF INVENTION

The present invention relates to an isolated population of osteoblastic cells, which support HSCs in vivo and in vitro. In particular, the present invention relates to an isolated population of osteoblastic cells, which are characterized by cell surface markers N-cad⁺ and CD45⁻. Such osteoblastic cells can be used in vitro to promote and support growth of HSCs. The present invention also relates to methods for isolating the osteoblast population, as well as methods for supporting HSCs in vitro. Related to this is a culture having HSC and N-cad⁺CD45⁻ osteoblasts. Additionally, the present invention relates to vectors, which include a Bmpr1a nucleic acid sequence, recombination sites, and a plasmid. Antibodies and Fab fragments to the Bmpr1a polypeptide are also contemplated. The vectors and antibodies can be used to promote an increase in the HSC population in vivo.

As mentioned, the isolated population of osteoblastic cells characterized by N-cad⁺CD45⁻ can be used for supporting and promoting growth of HSC in vitro. In particular, the osteoblasts can be used as a feeder layer in an in vitro culture. The osteoblast cells can be obtained by flushing bone marrow cells from tibias and femurs of a selected host into solution. A suitable solution is PBS; however, other solutions may be used, as long as the efficacy of the cells is maintained. Any of a variety of media and solutions can be used to maintain the integrity of the cells. As such, the femurs or tibias will be flushed until a sufficient population is obtained. Additionally, a bone sample from more than one specimen can be flushed to obtain a sufficient cell population. A sufficient amount of cells should be flushed to provide a suitable population for eventual use as feeder cells. Typically, a population equal to at least 1×10⁵ cells should be isolated. The population can be determined by using a cell counter. Such an amount is sufficient for at least one starter culture. If larger applications are to be practiced, obviously, a greater number of cells should be isolated.

An alternative to flushing the bone sample is to grind the bone. The ground bone is then treated as listed below. This is especially well suited with mice.

For humans, a bone marrow cell sample is obtained and cultured. This is done by removing marrow cells from the bone.

The population of cells isolated from the bone marrow sample will include hematopoietic and stromal stem cells. As such, the bone marrow will include osteoblasts, mesenchymal, endothelial, fibroblasts, and hematopoietic cells, and isolated stromal and hematopoietic stem cells. The hematopoietic cells will include lymphoid progenitor cells and myeloid progenitor cells. The stromal stem cells include osteoblasts. After isolation, the population of bone marrow cells is treated to remove the myeloid, more particularly, the red blood cells. The red blood cells can be lysed using ammonium chloride, for example. The remaining cell types are separated from the lysed red blood cells and are then ready for analysis. Included in the remaining cells are white blood cells, such as lymphocytes, leukocytes, as well as bone marrow cells, such as osteoblasts.

Once the white blood cells are separated from the red blood cells, the osteoblastic cells are separated from the remaining cells. The isolated cells are mixed with cell surface markers which enable separation of the osteoblast cells from the white blood cells and separation into discrete sub-populations. The cell surface markers are a variety of antibodies, which attach to the cell surface of a specific cell type. The antibodies are labeled with any of a variety of labeling compounds. Kits are commercially available, such as FITC-labeled lineage markers, APC-c-Kit, and PE-Sca-1. Other fluorescent cell surface markers may also be used. As is known, the antibody attaches to a specific antigen on the cell surface. In the present case, the fluorescent FITC-labeled antibodies to the cell surface markers N-cadherin (N-cad) and CD45 are mixed with the cells. After a period of incubation, the marked cells are passed through a flow cytometer, such as a FACS sorter, or similar device, whereby individual cells are separated into discrete populations so that the N-cad⁺CD45⁻ osteoblast cells are separated from the remainder of the osteoblastic cells. It should be noted that there are other ways to separate cells. This isolated population can then be used for any of a variety of applications, including in vitro support of HSC.

Thus, once a sufficient cell population is achieved, stromal cells are separated from the remainder of the culture. Mesenchymal stem cells are then separated from the stromal cells using a FACS sorter. BMP2 is induced in the mesenchymal stem cells and the resultant population is sorted. Osteoplastic cells are then induced and sorted.

As such, the present invention relates to a particular cell lineage identified by selected cell surface markers. Importantly, this population of spindle-shaped N-cadherin+CD45⁻ osteoblastic cells are different from the other osteoblasts, which are bone-matrix forming cells. The isolated N-cad⁺CD45⁻ osteoblastic cells can be used as niche cells for supporting HSCs. In particular, the osteoblasts can be used as feeder cells to expand stem cell populations in vitro. In addition, in vitro co-culture of HSCs with isolated osteoblasts will facilitate further dissection of molecular control of stem cell self-renewal in a relative high-throughput way.

The osteoblast cells can be derived from any of a variety of organisms, including a variety of different mammals. Available mammalian osteoblastic cells include mice, rats, humans, goats, rabbits, guinea pigs, and any of a variety of other mammals.

As mentioned, once isolated, the osteoblast cells are used as feeder cells to support HSC growth. The osteoblasts are added to a culture flask, for example, and then washed. Use as feeder cells is initiated by first plating the osteoblasts so that the N-cad⁺CD45⁻ cells are no longer dividing. Division is stopped by irradiating the cells. Media is then mixed with the osteoblasts to maintain the viability of the cells. The media can be formed from a variety of constituents, as long as the osteoblast cells are sufficiently supported. Importantly, after mixing the feeder cells must promote growth of the HSC, preferably Lin⁻Sca-1⁺, c-Kit⁺, CD45⁺, N-cadherin⁺ and BrdU-LTR HSC. The feeder cells are then, for example, plated. The HSCs are then added to the osteoblast feeder cells. The osteoblasts are attached to the solid phase surface of wells using poly-L-lysine, or a collagen. Standard conditions for the promotion of the HSC population increase are used, including standard temperatures. The HSCs are harvested once a sufficient population is achieved.

A typical feeder cell mixture includes DMEM and plasma F12 in a 1:1 ratio by volume. Added to the DMEM mixture is 10% FBS by volume. The N-cad⁺CD45⁻ osteoblasts are added to the composition. The osteoblasts are added after they are isolated, cultured, and irradiated. HSCs in a medium are then added to the osteoblasts mixture. The HSC medium will contain various amino acids, growth factors, and vitamins. It can contain, for example, IMDM, IL-3, TPO, and a cytokine kit.

HSCs derived from any of a variety of the above mentioned organisms can be mixed with the osteoblast cells. Different types of HSC may be mixed therewith; however, it is most preferred to use LT-HSC, specifically Lin⁻Sca-1⁺, c-Kit⁺, CD45⁺, N-cadherin⁺ and BrdU-LTR HSC.

The niche size must be tightly regulated in vivo to maintain HSCs and normal homeostasis. Observations from Bmpr1a conditional mutant mice strongly support this view and demonstrate the impact of the niche on the stability of the stem cell compartment. In vivo evidence in mammals is provided to show that change in the niche size, through genetic manipulation, affects the stem cell number. BMP signaling through Bmpr1a is a major genetic pathway involved in this regulation.

To promote increased HSC in vivo, any of a variety of methods is available for use. The known methods include using a knockout animal to eliminate the functionality of a selected gene. Other available options include using antibodies or related molecules to bind and inhibit a selected polypeptide.

Methods for formation and use of a knockout animal are well known. Use of the knockout animal is initiated by isolating a wt Bmpr1a gene or nucleic acid sequence, SEQ ID NO. 3. The sequence is amplified using PCR, for example. A mutation is made in the sequence, preferably before amplification. The mutation can be a frame shift, point, substitution, or deletion mutation. Importantly, the sequence should remain substantially homologous to the wt, but render the resultant gene non-functional. Next, the mutant sequence is used in forming a recombination vector. The vector will include the mutant Bmpr1a nucleic acid sequence, and is preferably flanked by recombination cites. The vector is structured such that the selected gene will eventually be cut from the genome and rendered inhibited. An alternative is to use a mutated version of the sequence so that the gene is rendered non-functional. As such, the wild type Bmpr1a gene sequence found in a selected host organism will be eliminated or made non-functional through recombination with the vector's mutant Bmpr1a nucleic acid sequence. Any of a variety of vectors may be used. SEQ ID NO. 2 is an example of a mutated Bmpr1a sequence that can be used in a recombination vector. The truncated Bmpr1a protein formed by SEQ ID NO. 2 is indicated in SEQ ID NO. 1.

More particularly, it is preferred to form a conditional transgenic Bmpr1a knockout mouse. This can be achieved by the knock-in of a Cre or Flp recombinase site (or a combination of these) into a specific gene locus (or two loci). The expression of Cre and Flp will be under the control of the endogenous locus in a tissue-specific, time-dependent manner. The temporal/spatial-restricted Cre/Flp expression line will lead to a selective, or conditional deletion of the gene of interest when crossed to a line of mice in which LoxP or FRT recognition sites are made to flank the gene of interest. In addition, a combination of the Cre/LoxP and Flp/FRT systems will allow selective and simultaneous deletion of two loci of interest.

The Cre-LoxP recombination system can be used to create transgenic mice with cell-specific deletions of the Bmpr1a gene. Gene targeting of the Bmpr1a gene in ES cells with a LoxP replacement vector will be used to generate a heterozygous flox-Bmpr1a founder mouse that can be crossed to a homozygous Cre mouse.

The only two functional units required for in vivo targeted DNA deletion with the Cre-LoxP system are: 1) expression of the P1 Cre recombinase gene, often times by a cell-specific or regulated promoter; and 2) an integrated DNA segment that is flanked by direct repeat copies of the 34 bp P1 DNA sequence called LoxP. LoxP-targeted DNA is said to be “floxed.”

The Cre/LoxP system is a tool for tissue-specific and time specific knockout of selected genes, which cannot be investigated in differentiated tissues because of their early embryonic lethality in mice with conventional knockouts. It can also be used for the removal of a transgene (which was overexpressed in a specific tissue) at a certain time point to study the inverse effect of a down-regulation of the transgene in a time course experiment.

Thus, two mouse lines are required for conditional gene deletion. First, a conventional transgenic mouse line with Cre targeted to a specific tissue or cell type, and secondly, a mouse strain that embodies a target gene (endogenous gene or transgene) flanked by two LoxP sites in a direct orientation (“floxed gene”). The target gene described herein is the Bmpr1a gene. Recombination (excision and, consequently, inactivation of the target gene) occurs only in those cells expressing Cre recombinase. Hence, the target gene remains active in all cells and tissues which do not express Cre.

In addition to gene deletion methods in vivo, an antibody to a gene product can be used to generate phenotypic changes in a host organism. Thus, an antibody to the Bmpr1a polypeptide may also be used. Such antibody will prevent the functioning of the Bmpr1a polypeptide and, thus, result in an increase in the HSC population in vivo.

The mechanisms involved in controlling the number of adult stem cells remain largely unknown. The present invention demonstrates that change in niche size affects the number of hematopoietic stem cells (HSCs). By analyzing mutant mice with conditional inactivation of BMP receptor type IA (Bmpr1a), it was found that the number of HSC was increased, as evidenced by immunophenotypical and functional assays. Intriguingly, the increase in the HSC number correlates with an increase in the trabecular bone volume and with an increase in the number of spindle-shaped N-cadherin⁺CD45⁻ osteoblastic cells. Long-term HSCs (Lin⁻Sca-1⁺c-Kit⁺CD45⁺N-cadherin⁺) were found to be attached to the spindle-shaped N-cadherin⁺CD45⁻ cells located on the bone surface. Furthermore, N-cadherin and β-catenin, two adherens-junction molecules, are asymmetrically localized to the cell surfaces between long-term HSCs and the spindle-shaped osteoblastic cells. It was concluded that the N-cadherin⁺CD45⁻ osteoblastic cells lining the bone surface function as a key component of the niche to support HSCs. Thus, BMP signaling, through the Bmpr1a receptor, controls the number of HSCs via regulation of niche size.

The following definitions define terms used herein:

Allele is a shorthand form for allelomorph, which is one of a series of possible alternative forms for a given gene differing in the DNA sequence and affecting the functioning of a single product.

An amino acid (aminocarboxylic acid) is a component of proteins and peptides. Joining together of amino acids forms polypeptides. Polymers containing 50 or more amino acids are called proteins. All amino acids contain a central carbon atom to which an amino group, a carboxyl group, and a hydrogen atom are attached. Protein molecules can be referred to as polypeptides when the protein molecule is less than 500 amino acids in length.

An antigen (Ag) is any molecule that can bind specifically to an antibody (Ab). Their name arises from their ability to generate antibodies. Each Ab molecule has a unique Ag binding pocket that enables it to bind specifically to its corresponding antigen. Abs are produced by B cells and plasma cells in response to infection or immunization, bind to and neutralize pathogens, or prepare them for uptake and destruction by phagocytes.

BMP—Bone morphogenic protein.

Bmpr1a—bone morphogenetic protein receptor, type 1A, expressed almost exclusively in skeletal muscle. Bmpr1a is a regulator of chondrocyte differentiation, down stream mediator of Indian Hedgehog, TGFBR superfamily, and activin receptor-like kinase 3.

Bone marrow is defined as a soft, highly vascular modified connective tissue that occupies the cavities and cancellous part of most bones and occurs in two forms: 1) a whitish or yellowish bone marrow consisting chiefly of fat cells and predominating in the cavities of the long bones—called also yellow marrow; and, 2) a reddish bone marrow containing little fat, being the chief seat of red blood cell and blood granulocyte formation, and occurring in the normal adult only in cancellous tissue, especially in certain flat bones—also called red marrow.

The bone marrow is the main site of hematopoiesis, the generation of the cellular elements of blood, including red blood cells, monocytes, polymorphonuclear leukocytes, and platelets. The bone marrow is also the site of B-cell development in mammals and the source of stem cells that give rise to T cells upon migration to the thymus. Thus, bone marrow transplantation can restore all the cellular elements of the blood, including the cells required for adaptive immunity.

CD is the name for commonly used cell surface Ags that are useful in discriminating between different cell types. The cell-surface molecule is designated CD followed by a number (e.g., CD3, CD4, CD8, etc.) The CD3 complex contains the Ag-specific T cell receptor (highly variable between different T cell populations). This receptor is responsible for Ag recognition by forming Ag binding pockets analogous to those of Ab molecules. CD4 is a marker expressed on helper T cells. CD8 is associated with cytotoxic T cells. The CD4 and CD8 molecules, themselves, appear on different T cells. CD34 is defined as a cell surface marker. CD stands for cluster of differentiation and the 34+indicates a specific antigen for which this cell is positive. Stem cells are CD34⁺.

Chimera is an individual composed of a mixture of genetically different cells. The genetically different cells of chimeras are required to be derived from genetically different zygotes.

DNA cassette is a deoxyribonucleic acid (DNA) sequence that can be inserted into a cell's DNA sequence. The cell in which the DNA cassette is inserted can be a prokaryotic or eukaryotic cell. The prokaryotic cell may be a bacterial cell. The DNA cassette may include one or more markers, such as Neo and/or LacZ. The cassette may contain stop codons. In particular, a Neo-LacZ cassette is a DNA cassette that can be inserted into a cell's DNA sequence located in a bacterial artificial chromosome (BAC). Such DNA cassettes can be used in homologous recombination to insert specific DNA sequences into targeted areas in known genes.

Ectopic is defined as an overexpression of a gene or a site other than the native site.

FACS is defined as fluorescent activated cell sorter.

Feeder Layer is a layer of cells (usually irradiated to prevent cell proliferation for animal cell culture) upon which are cultured a fastidious cell type.

Flow cytometry is defined as the analysis of biological material by detection of the light-absorbing or fluorescing properties of cells or subcellular fractions (i.e., chromosomes) passing in a narrow stream through a laser beam.

A gene is a hereditary unit that has one or more specific effects upon the phenotype of the organism that can mutate to various allelic forms.

HSC is defined as hematopoietic stem cell, including LT-HSC and ST-HSC A host organism is an organism that receives a foreign biological molecule, including an antibody or genetic construct, such as a vector containing a gene.

Knockout is an informal term coined for the generation of a mutant organism (generally a mouse) containing a null allele of a gene under study. Usually the animal is genetically engineered with specified wild-type alleles replaced with mutated ones. Knockout also refers to the mutant animal.

Mutation is defined as a phenotypic variant resulting from a changed or new gene.

Mutant is an organism bearing a mutant gene that expresses itself in the phenotype of the organism. Mutants include both changes to a nucleic acid sequence, as well as elimination of a sequence or a part of a sequence. In addition polypeptides can be expressed from the mutants.

A nucleic acid is a nucleotide polymer better known as one of the monomeric units from which DNA or RNA polymers are constructed, it consists of a purine or pyrimidine base, a pentose, and a phosphoric acid group.

Osteoblasts are cells from which bone develops.

Osteoclasts are large multinuclear cells that resorb bony tissue in osteoclasis.

Plasmids are double-stranded, closed DNA molecules ranging in size from 1 to 200 kilo-bases. Plasmids are incorporated into vectors for transfecting a host with a nucleic acid molecule.

A polypeptide is a polymer made up of less than 50 amino acids.

Small molecules are defined as regulatory polypeptide or nucleic acid molecules that cause detectable changes in protein-protein interaction systems that may also affect one or more phenotypic changes. These small molecules may operatively function by structural similarity to and competitive inhibition with native molecules in vitro or in vivo. Phenotypic changes may include observed changes in such parameters as HSC-proliferation, bone deposition or bone mineral density, tooth development, and ocular development. Small regulatory polypeptide molecules include, but are not limited to, antibody fragments such as Fab, F(ab)₂, Fv, and antibody combining regions. Small regulatory nucleic acid molecules include, but are not limited to, antisense RNA sequences that interfere with wild type polypeptide function; and truncated nucleic acid sequences that encode shortened polypeptides that interfere with function.

A stem cell is defined as a pluripotent or multipotent cell that has the ability to divide (self-replicate or self-renew) for indefinite periods—often throughout the life of the organism. Under the right conditions, or given the right signals, stem cells can give rise (differentiate) to the many different cell types that make up the organism. Stem cells have the potential to develop into mature cells that have characteristic shapes and specialized functions, such as blood cells, heart cells, skin cells, or nerve cells.

A stem cell marker is defined as a specialized protein (receptor) on the surface of every cell in the body that has the capability of selectively binding or adhering to other “signaling” molecules. There are many different types of receptors that differ in their structure and affinity for the signaling molecules. Typically, the stem cell marker is an antigen.

Support is defined as establishing viability, growth, proliferation, self-renewal, maturation, differentiation, and combinations thereof, in a cell. In particular, to support an HSC population refers to promoting viability, growth, proliferation, self-renewal, maturation, differentiation, and combinations thereof, in the HSC population. Support of a cell may occur in vivo or in vitro.

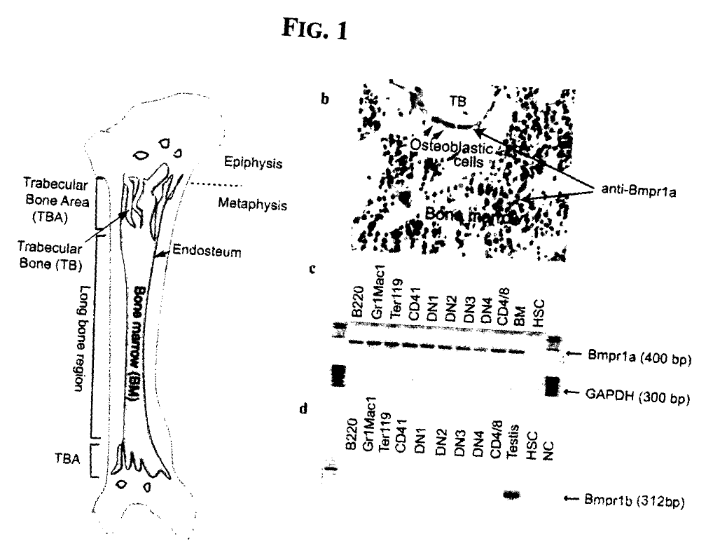

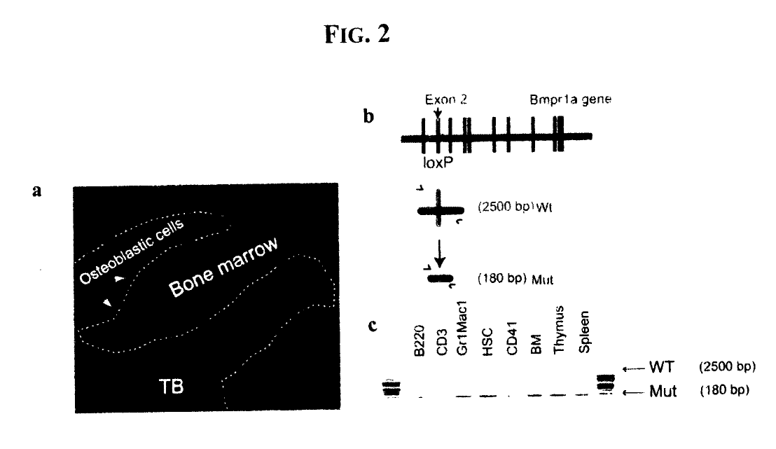

TBA means a trabecular bone area, shown in FIG. 1a.

TBLA means a trabecular bone-like area in mutants.

A vector is a self-replication DNA molecule that transfers a DNA segment to a host cell.

Wild type is the most frequently observed phenotype, or the one arbitrarily designated as “normal”. Often symbolized by “+” or “wt.”

As will be shown, a small subset of ostoblastic cells, which have a similar rarity (0.05%) as that of long term HSCs (0.01-0.05%) in bone marrow, have been identified. This means that the existence of such a population of niche cells is either absent in other types of stromal cells, including fibroblasts, endothelial cells, adipocytes, and macrophages, or is a very limited portion of the osteoblastic cell line.

Human BMPR1A (ACVRLK3; ALK3)—Genecard No. GC10P087738: Bone Morphogenetic Protein Receptor, Type 1A, also known as Activin A Receptor, Type II-like Kinase 3. The chromosomal location of Bmpr1a in the human genome is at LocusLink Cytogenetic Band: 10q22.3; ENSEMBL Cytogenetic Band 10q23.2. The GeneLoc location for Bmpr1a starts 87,738,131 bp from pter and ends 87,906,650 bp from pter. The size of the gene is 168,519 bases and it is oriented in a plus strand. Additional cDNA sequences of interest have the following locus identification numbers BC028383 and Z22535.

Human BMPR1A mRNA—The LocusLink identification for the human BMPR1A mRNA is NM_{—}004329. The mRNA is 2932 base pairs long and is linear.

BMRA_Human: Protein—GDB (Genome Database) 230245: BMRA is comprised of 532 amino acids and has a molecular weight of 60201 Da. The BMRA protein functions as a receptor for BMP-2 and BMP-4. BMRA is highly expressed in skeletal muscle and heterodimerizes with a Type-II receptor. It belongs to the ser/thr family of protein kinases in the Tumerogenesis Growth Factor B (TGFB) receptor subfamily.

Mouse BMPR1A—GENBANK ID No. BB616238 is a mouse est similar to human LOC88582 (BMPR1A related). The UniGene ID No. is Mm.140965. This is a Bone Morphogenetic Protein Receptor, Type 1a sequence which genemapps to the cell line XC131 protein accession number XP_{—}017633 mouse chromosome.

BMRA_MOUSE: The primary accession number for the BMRA mouse protein is P36895. The protein is named Bone Morphogenetic Protein Receptor Type 1a. This protein is also known as EC2.7/1/37, Serine/threonine-protein kinase receptor R5, SKR5, Activin Receptor-like Kinase 3, ALK-3, BMP-2/BMP-4 receptor.

Homologues to BMPR1A in other animals include the following:

1. Rat, about 82% homologous to the human gene, has LocusLink number 81507 and NCBI accession number NM_{—}030849.1.

2. Mouse homologues have NCBI accession numbers AU045487, D16250, U04672, U04673, and Z23154. Another mouse homolog, about 86% to the human gene, has LocusLink number 12166 and NCBI accession number NM_{—}009758.1.

3. Frog (Xenopus), about 77% homologous to the human gene, has NCBI accession number D32066.1.

4. Zebrafish homolog, about 77% homologous to the human gene, has NCBI accession number AB011826.1 and LocusLink number 58145.m

5. A C. elegans homolog, about 44.5% homologous to the human gene, has NCBI accession number CB280190.1.

### EXAMPLES

### Example 1

An investigation of the roles of the BMP signaling pathway in regulating adult HSC development in vivo was done. An approach was taken of blocking the BMP signal through inactivation of one of its receptors and assaying the consequences in the corresponding animal model. More particularly, the expression patterns of Bmpr1a (Alk3) and b (Alk6) were analyzed in bone marrow and the surrounding bone tissue. Expression of Bmpr1a was found, by immunohistochemical staining (IHS) in bone marrow cells and most osteoblasts using anti-Bmpr1a antiserum, shown in FIG. 1b. As such, bone/bone marrow sections from Wt C57BL/6 mice were immunohistochemically stained using anti-Bmpr1a serum.

Using an RT-PCR assay, expression of Bmpr1a was detected in most, if not all, hematopoietic lineages apart from the HSC population, shown in FIG. 1c. To examine expression GAPDH (glyceraldehyde-3-phosphate dehydrogenase) was used as an internal control. The various hematopoietic cell types shown in the FIGS. 1c and 1d were as follows: B220 is a B cell marker; Gr1Mac1 is a Myeloid lineage marker; Ter119 is an erythroid lineage marker; CD41 is a megakaryocyte marker; DN is a double negative for CD4 and CD8; DN1 is a CD44⁺CD25⁻; DN2 is a CD44⁺CD25⁺ cell; DN3 is a CD44⁻CD25⁺ cell; DN4 is a CD44⁻CD25⁻ cell; BM is bone marrow; and, HSC is a hematopoietic stem cell. In contrast, expression of Bmpr1b in HSCs and the other hematopoietic lineages was undetected, as is shown in FIG. 1d. Thus, Bmpr1a is formed in bone marrow and osteoclasts cells, as well as hematopoietic lineages.

### Example 2

It is known that mice with a deletion of Bmpr1b are viable but have not been reported to have hematopoietic defects, while mice with the null Bmpr1a mutation are embryonically lethal. Thus, direct investigation of BMP's role in the late stages of development is difficult. A conditional knockout mouse model was generated by crossing a Bmpr1a^{fx/fx} mouse line with an interferon-inducible Mx1-Cre mouse line. In the Bmpr1a^{fx/fx} mouse line, Exon 2 of the Bmpr1a gene is flanked by two LoxP sites, shown in FIG. 2b, and can, therefore, be excised by Cre-mediated recombination. Using PCR analysis, pre-excision (2500 bp) and post-excision (180 bp) of Exon 2 can be determined. Heterozygous Bmpr1a^{+/-} was also used to generate Bmpr1a^{fx/-} as a control. Efficiency of Mx1-Cre in inducing recombination was examined by crossing Mx1-Cre mice with Z/EG reporter mice. The mice were then assayed following interferon induction by injection of polyl:polyC (PolyI:C, 250 μg/mouse). In general, only 50% of bone marrow cells (as measured by flow cytometric assay) revealed successful targeting as evidenced by expression of GFP following a single injection of PolyI:C (see also FIG. 2a). It was found that multiple injections of PolyI:C generated higher efficiencies.

Next, Bmpr1a^{fx/fx}, Bmpr1a^{fx/-}, Bmpr1a^{fx/+}, and Mx1-Cre mice were crossed to produce Mx1-Cre⁺Bmpr1a^{fx/fx} and Mx1-Cre⁺Bmpr1a^{fx/-} homozygote (Bmpr1a mutant), Mx1-Cre⁺Bmpr1a^{fx/+} and Mx1-Cre-Bmpr1a^{fx/-} heterozygote, as well as Mx1-Cre-Bmpr1a^{fx/fx}, which displayed a wild type (Wt) phenotype. Injection of PolyI:C induced excision of Exon 2 of the Bmpr1a gene mediated by the flanked LoxP sites, illustrated at FIG. 2b. Successful targeting was monitored by PCR and judged by the change in the sizes of the PCR product from 2500 bp (Wt) to 180 bp (Mut), as shown in FIGS. 2b and 2c. The Bmpr1a locus was successfully targeted following three injections of PolyI:C at an interval of every other day, as shown by PCR results obtained after three months, as shown in FIG. 2c.

Mice with different injection schedules were investigated. For the early-induced group, PolyI:C was injected on the 3rd, 5th, and 7th day after birth, while the late-induced group was treated with PolyI:C on the 21st, 23rd, and 25th day after birth. A third double-induced group combined both injection schedules. Analyses were performed one month after the last PolyI:C injection. At autopsy, no macroscopic signs of enlarged or atypical spleen, thymus, or lymph nodes were found.

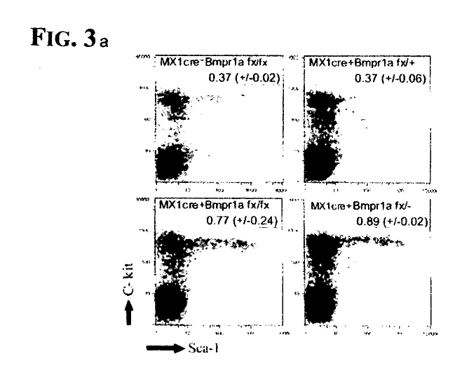

Flow cytometric assays were performed to analyze the HSC population in Bmpr1a mutant mice. The percentage of lineage-negative (Lin⁻) Sca-1⁺C-kit⁺ (HSC) cells was increased 2-fold in the Mx1-Cre⁺Bmpr1a^{fx/fx} and 2.4-fold in the Mx1-Cre⁺Bmpr1a^{fx/1} mutant mice, as compared to the littermate controls, as shown in FIG. 3a. Since the total bone marrow mononuclear cell number per femur was reduced in both of the Bmpr1a mutant lines due to reduced cavity room, the absolute number of HSCs per femur was still increased 1.5-2 fold in both early and late-induced knockouts, as shown in FIG. b. The total bone marrow cell number in the Bmpr1a mutant mice was reduced (average 1.58×10⁷ versus 2.1×10⁷ in Wt control mice). The absolute HSC number was determined by the percentage of HSCs×total mononuclear BM cells. No significant phenotypic difference was observed between Mx1-Cre⁺Bmpr1a^{fx/fx} and Mx1-Cre⁺Bmpr1a^{fx/-} mutants.

### Example 3

HSC populations of Mx1-Cre⁺Bmpr1a^{fx/fx} mutant mice were further analyzed. As is known, an HSC population is a heterogeneous mixture, including long-term (LT) and short-term (ST) HSCs. To distinguish LT-HSC from ST-HSC, mice were injected with BrdU to label cycling cells. Three hours after labeling, the HSC population was analyzed to distinguish ST-HSCs from LT-HSCs, according to the differences in their cell-cycle state. The percentage of BrdU⁻ HSC population (including the quiescent LT-HSCs) increased by an average of 2.4 times in the Bmpr1a mutant mice, as compared to littermate controls. The percentage of BrdU-positive HSC population (including the cycling ST-HSCs) was similar to that in the littermate controls, as shown in FIG. 3c. Therefore, direct comparison of the entire HSC population provided an under-estimated, but reliable representation, shown in FIG. 3b (1.6 fold) of the LT-HSC number difference, shown in FIG. 3c (2.4 fold), between the mutant and control animals. This measure of HSC percentages was used in the studies below.

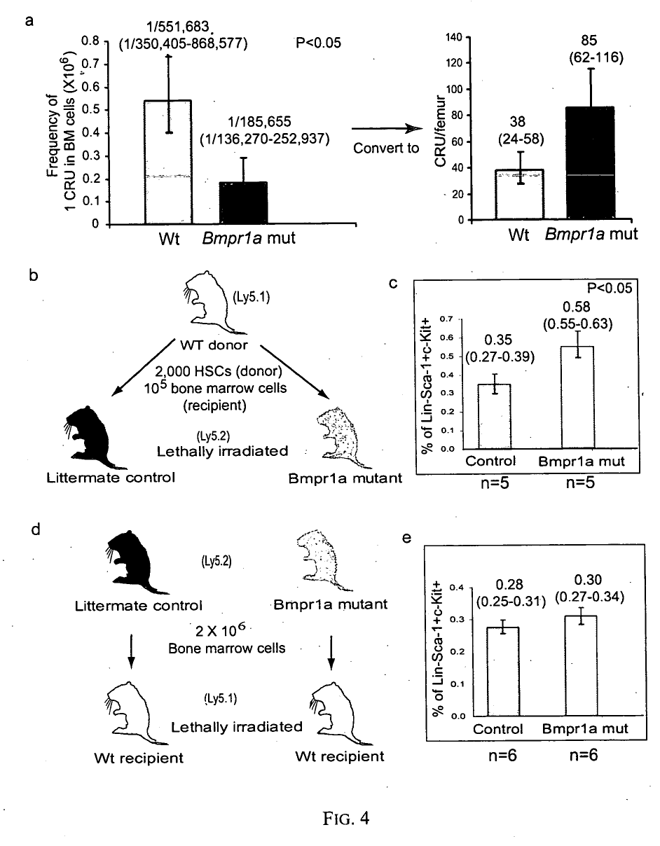

Measurement of BrdU-negative HSC population indicated an increase in the LT-HSC population. However, a preferred test is the competitive repopulation unit (CRU) assay, in which a series of diluted donor derived bone marrow mononuclear cells are transplanted into different groups of sublethally irradiated recipient mice. Three months after transplantation, peripheral blood was examined to monitor the engraftment of donor cells in the recipient mice. The lowest number of bone marrow mononuclear cells required for reconstitution of hematopoiesis in the sublethally irradiated recipient mice was determined. The CRU in bone marrow mononulcear cells was calculated using the L-Calc software. Using this assay, it was confirmed that the functional stem cell number increased 2.2-fold in the mutant mice compared with that of Wt control, as shown in FIG. 4a. CRU/femur (right panel)=frequency of CRU (left panel)×total mononuclear BM cells (average 1.58×10⁷ in the Bmpr1a mutant mice versus 2.1×10⁷ in Wt control mice), the frequency of CRU is determined by the lowest number of bone marrow cells that are required to reconstitute recipient hematopoietic system in transplantation experiments with a series of diluted done bone marrow cells. The ranges of experimental data are listed in parentheses. This was consistent with the result from the flow cytometric assay of LT-HSCs, shown in FIG. 3c. The results from both immunophenotypical and CRU assays indicated that the population of LT-HSCs is expanded in the Bmpr1a mutant mice.

### Example 4

There are several mechanisms which can lead to changes in the HSC population number: 1) an intrinsic change in stem cells that either promotes self-renewal or blocks apoptosis; 2) an internal defect in progenitors that inhibits differentiation, leading to an accumulation of stem cells; or 3) an external influence from the HSC microenvironment. As such, the mice and cells were further analyzed to determine the cause of the change in the HSC number.

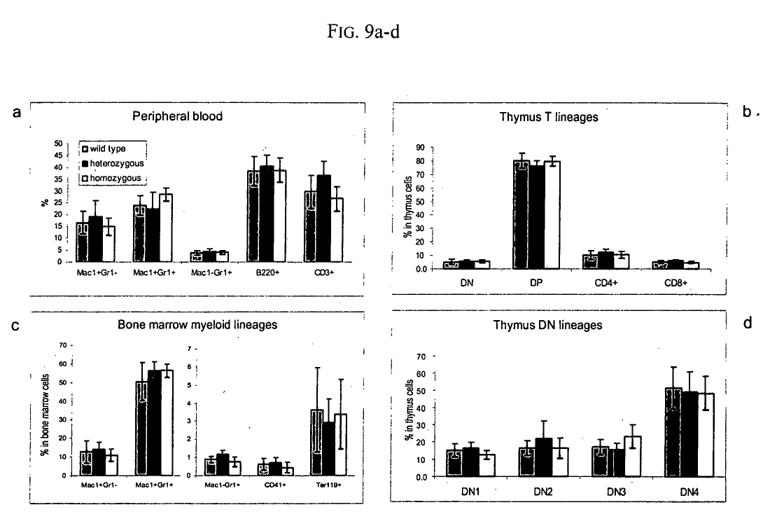

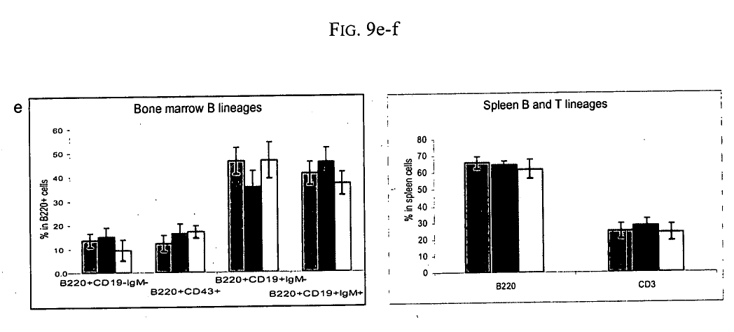

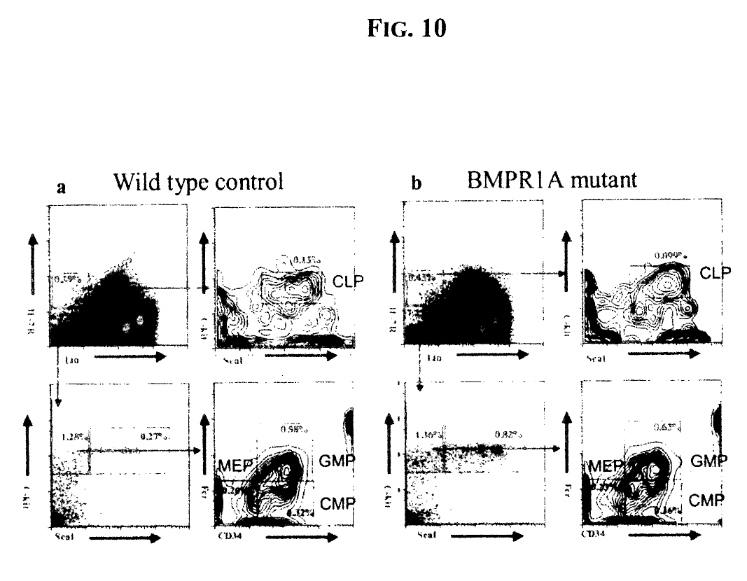

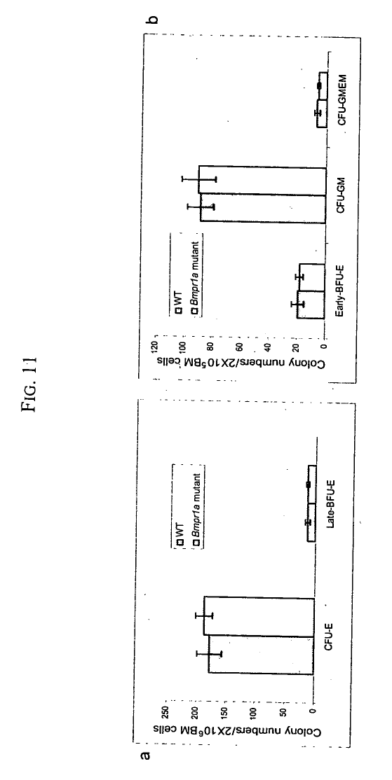

The myeloid and lymphoid lineages were analyzed in peripheral blood, thymus, spleen and bone marrow of Bmpr1a mutant and wild type mice. These lineages were the common myeloid progenitor (CMP) and common lymphoid progenitor (CLP) subsets found in the bone marrow. The results revealed no significant phenotypical change in any of the subsets analyzed, as shown in FIG. 9. One month after polyl:C treatment (3 times), bone marrow, thymus and spleen mononuclear cells were collected and stained with different lineage cell surface markers. Gr1, Mac1, B220, and CD3 for peripheral blood cells; Gr1, Mac CD41, and Ter119 for bone marrow myeloid cells; B220, CD3, IgM, CD19, and CD43 for bone marrow lymphocytes; B220 and CD3 for spleen; CD3, CD4, CD8 for mature T cells; and, CD3, CD4, CD8 co-staining with CD25, CD44 for T lymphocyte progenitors in thymus. DN relates to CD4CD8 double negative cells DN1-4 were defined in FIG. 1c. The percentages of progenitors and different mature blood cell lineages were analyzed by BD LSR flow cytometry. No significant difference was found among wild type control heterozygous and homozygous Bmpr1a mutant mice in all the lineages analyzed, as well as among common lymphocyte progenitor (CLP) and common myeloid progenitor (CMP).

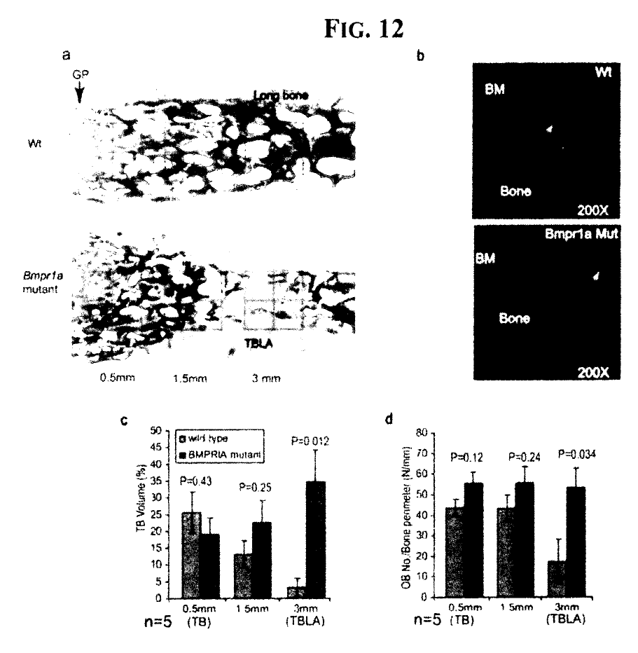

The percentages of each population, including GMP and MEP, are indicated in the corresponding gate of FIG. 10. A flow cytometry assay was performed following the procedures disclosed herein. In the methods, HSC and hematopoietic progenitors were enumerated using modifications of a published procedure. In brief, bone marrow cells were flushed from femurs into PBS with 1% FBS and cell concentrations were determined using a Hemavet cell counter. Red blood cells were transferred to small Eppendorf tubes containing precisely titrated antibody cocktails (all antibodies—eBioscience, CA). Color compensation samples were produced by singly staining bone marrow WBC with one antibody of each of the 6 fluorochromes. Other controls for the assay were a combination of cells stained by a method called Fluorescence Minus One (FMO) and also by the use of isotope antibodies. These antibodies were: IgG2b-FITC, IgG-FITC IgG2a-Pe, IgG2a-PeCy5, IgG1-Apc, and IgG2b-Biotin. The FMO method was performed by sequentially adding fluorescent labeled antibodies to the staining cocktail. For example, the first of the FMO tube contained only B220-FITC, Ter119-FITC, CD3-FITC, IgM-FITC, and Gr1-FITC antibodies (Lin-FITC). The second and third FMO tubes contained Lin-FITC plus IL7ra-PeCy5 or its Pe-Cy5 isotype. The forth and fifth FMO tubes contained Lin-FITC and IL7ra-PeCy5, with the addition of Sca1-Pe, and c-Kit-Apc, with the addition of CD16/32-PeCy7 and IgG2b-Biotin. Biotin antibodies were further incubated with Strepavidin-ApcCy7. The final assay tube contained Lin-FITC, Sca1-PE, IL7ra-PeCy5, CD16/32-PeCy7, c-Kit-APC, and CD34-Biotin-Strepavidin ApcCy7. All data was collected on a MoFlo equipped with Coherent lasers producing 488 nm and 647 nm lines, or a Cyan equipped with a Coherent Enterprise and a solid state 635 nm laser (DakoCytomation, Fort Collins, Colo.). Data analysis was performed using FloJo software (TreeStar). This was consistent with results obtained from in vitro colony forming unit (CFU) assays. The results from CFU-GM (granulocyte/monocyte), BFU-E (pro-erythrocyte), CFU-E (pre-erythrocyte), and CFU-GMEM (granulocyte/monocyte/erythrocyte/megakaryocyte) indicated that the number of different lineage progenitor cells was similar in the Bmpr1a mutant and littermate control mice, shown in FIG. 11. Bone marrow mononuclear cells were collected 1 month after polyI:C treatment. Different stages of erythroid progenitors are represented by early BFU-E, late BFU-E and CFU-E, and granulocyte/monocyte progenitor (CFU-GM). Progenitors for all myeloid lineages (CFU-GMEM) were incubated and counted by following the protocol supplied by Stem Cell Technologies (Canada). These results ruled out the possibility of an accumulation of HSCs resulting from a block in progenitor cell differentiation.

Next, a test to determine whether the lack of change in hematopoietic differentiation was due to a growth advantage of Wt cells over the mutant cells was conducted. Three months after PolyI:C injection, at which time most hematopoietic cells were presumably replaced by newly generated cells derived from HSCs, the genomic DNA was analyzed, using a PCR method that could differentially amplify the mutated or Wt allele of the Bmpr1a gene. 2×10⁶ bone marrow mononuclear cells derived from either Bmpr1a mutant or Wt littermate control mice were transplanted into lethally irradiated Wt Ly5.1 mice. The analysis was carried out 3 months post-transplantation. It was found that the majority of hematopoietic cells examined carried the mutant allele, shown in FIG. 4d, ruling out the possibility of a growth advantage of Wt cells over the mutant cells.

### Example 5

External influences were analyzed to determine the effect on the HSC population. Direct evidence of an external influence causing an increase in the HSC number came from reciprocal bone marrow/HSC transplantation experiments. HSCs isolated from Wt Ly5.1 mice were transplanted into lethally irradiated Bmpr1a mutant and littermate control mice (both were Ly5.2 genotype), as shown in FIG. 4b. The lethally irradiated Bmpr1a mutant and Wt littermate control mice were transplanted with 2,000 HSCs (Lin⁻c-Kit⁺Sca-1⁺) per mouse derived from Wt Ly5.1 donor mice plus 1×10⁵ recipient derived bone marrow cells (for rescue), and the analysis was carried out 3 months post transplantation. The result from flow cytometric assay, three months after transplantation, revealed that the percentage of donor-derived HSCs in the Bmpr1a mutant mice was an average of 1.6 times higher than in the Wt control recipient mice, shown in FIG. 4b. A reciprocal assay was also carried out by transplanting bone marrow mononuclear cells derived from the Bmpr1a mutant or Wt control mice, into lethally irradiated Wt Ly5.1 mice, shown in FIG. 4d. These cytometric results showed that the percentage of donor derived HSCs in both groups of recipient mice was similar, regardless of the origin of bone marrow mononuclear cells, as shown in FIG. 4d. Taken together, it was concluded that the change in the microenvironment resulted in the increase in HSC number in the Bmpr1a mutant mice.

### Example 6

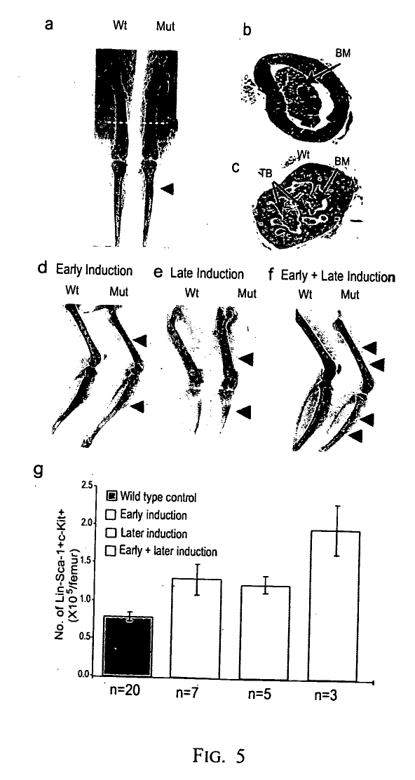

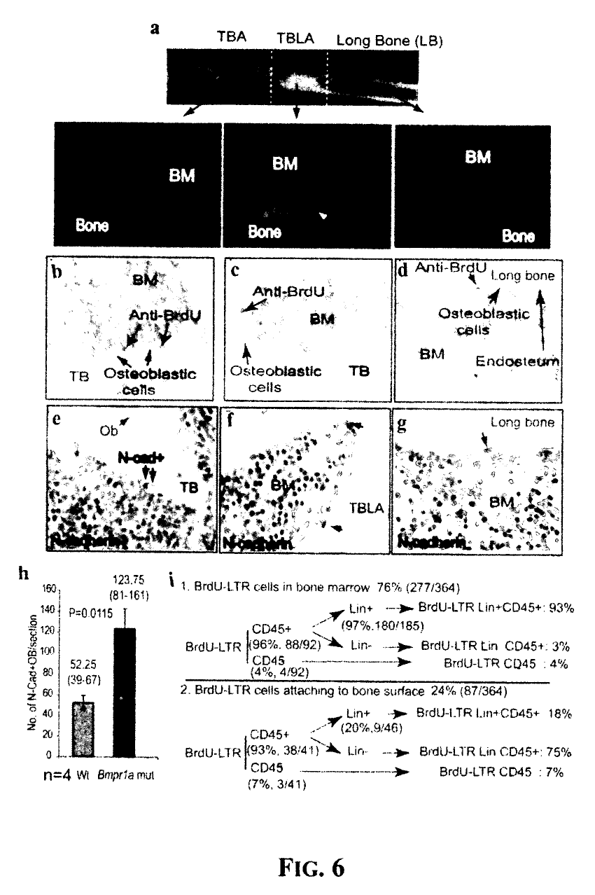

It was found that there was observable abnormal bone formation in the Bmpr1a mutant mice. The x-ray and histological analyses revealed that an ectopic formation of trabecular bone-like area (TBLA) occurred in the long bone region of the mutant mice, shown in FIGS. 5a-5c. A number of vesicles were found in the ectopically formed TBLA and these vesicles were usually found in the epiphysis region. The location of the ectopically formed TBLAs varied depending on the PolyI:C injection time. TBLAs were seen distal to the knee in the early-induced group, shown in FIG. 5d but were proximal to the knee in the late-induced group, shown in FIG. 5e, and at both sites in the combined injection group, as shown in FIG. 5f. This raised the possibility of a change in osteoblasts or osteoclasts occurring in the Bmpr1a mutant mice, which may affect the microenvironment for HSCs. Recent evidence showed that an osteoblastic cell line can increase the number of HSCs 2-4 fold in an in vitro assay of long-term culture-initiating cells (LTC-IC). In addition, osteoblasts, when co-transplanted with HSCs, can also increase the engraftment rate. These observations suggest that osteoblasts may play a role in supporting HSCs.

A question was raised as to whether the ectopically formed TBLA was responsible for the increased HSC number in the mutant mice. As described above, as shown in FIG. 3b, the number of HSCs per femur in the mutant animals was increased an average of 1.67 fold (ranging from 1.46 to 2-fold) in both the early and late-induced mutant mice, shown in FIG. 5g. In addition, it was found that the number of HSCs per femur increased an average of 2.5-fold (ranging from 2.1 to 3.0-fold) in the combined early and late-induced mice, shown in FIG. 5g, in which two regions of TBLA were observed, as shown in FIG. 5f. Thus, it was determined that an increase in the number of HSCs correlated with an increase in the number of ectopically formed TBLAs.

### Example 7

An investigation was performed regarding the mechanism that leads to the ectopic formation of TBLA when the BMP signal is blocked. Bmpr1a has been shown to inhibit osteoblastic lineage commitment from mesenchymal progenitors in vitro. The CFU-F (fibroblast) assay revealed that there was no difference in the count between Wt control and Bmpr1a mutant mice, ruling out the possibility of an increase in the number of mesenchymal progenitors in the mutants. An increase in osteoblastic lineage commitment from mesenchymal progenitors was not possible since ectopic formation of TBLA is only regional; otherwise, they should be evenly distributed. This led to the hypothesis of a regional inactivation of Bmpr1a leading to ectopic formation of TBLA. To confirm this, a triple-genotype mouse line was generated, which bears Mx1-Cre, Bmpr1a^{fx/fx}, and Z/EG alleles. As shown by FIG. 6a, Cre-induced GFP expression reflects successful targeting of Bmpr1a. Analysis of bone sections derived from the triple-genotype mice revealed that only the surface of ectopically formed TBLA was GFP positive, indicating that regional deletion of Bmpr1a occurs exclusively in the cells lining the TBLA surface. These observations supported the hypothesis, which was further supported by a 3-fold increase in osteoblast numbers, a 2-fold increase in the bone formation rate and a 10-fold increase in bone volume in the TBLA of Bmpr1a mutants, compared to the long bone region in control littermates.

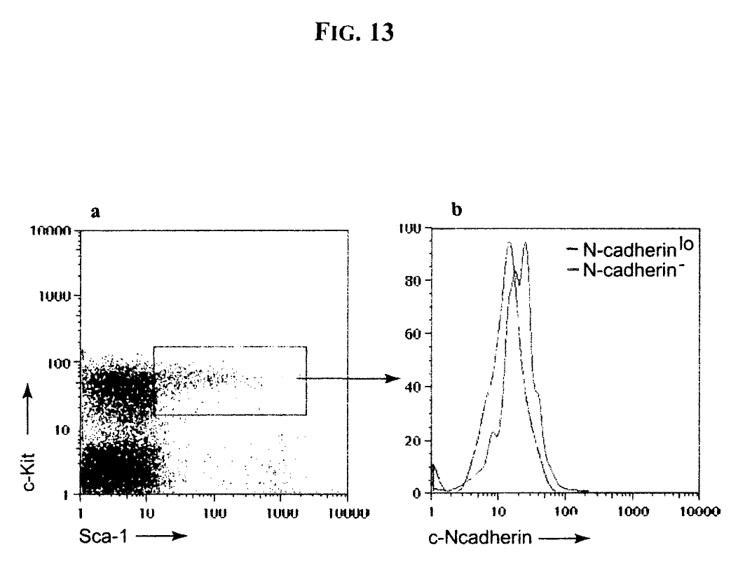

In FIG. 12, five pairs of femurs were collected from 6-week old Wt control and the Bmpr1a mutant mice, were fixed, decalcified, dehydrated, and embedded following the procedure described. Serial longitudinal section (4 μm thick) were prepared and stained with Hematoxyin-eosin (HE). Histomorphometry of bone sections was performed at three sites of the secondary spongiosa (0.5 mm, 1.5 mm, and 3 mm [the site with TBLA] from the growth plate); four optical fields from each site were analyzed using OsteoMeasure (OsteoMetrics Inc.) at 200× magnification. All sections were examined blindly. Both oval-shaped and spindle-shaped osteoblasts were counted in this study for the osteoblast quantitative analysis. Photos are shown at FIGS. 12a and 12b.

For bone formation rate fluorochrome labeling, 2-month old mice received an intraperitoneal injection of tetracycline (15 mg/kg body weight) on day 0 and calcein (20 mg/kg body weight) on day 6. Mice were sacrificed on day 8. Femurs were fixed in 99.5% ethanol and embedded in methylmethacrylate without decalcification. Serial longitudinal sections (7 μm thick) were prepared using a microtome. The sections were stained with Villanueva Goldner to discriminate between mineralized and unmineralized bone and to identify cellular components. Images were visualized by fluorescent light microscopy. Whereas, there is no apparent change in osteoclasts (cells reabsorbing bone), measured by TRAP (tartrate-resistant-acid-phosphatase) staining. All this data indicates that the ectopically formed TBLA occurs through either over-proliferation or abnormal differentiation of osteoblast progenitor. FIG. 12 illustrates the histomorphometry of bone sections, shown in FIG. 12a, bone forming rate, shown in FIG. 12b, measurement of trabecular bone volume, shown in FIG. 12c, and osteoblast number, shown in FIG. 12d.

### Example 8

A correlation between the number of LT-HSCs and the increase of TBLA was observed. It was desired to determine whether the LT-HSCs in the TBLA were enriched. The limited number of LT-HSCs in bone marrow, and the lack of a unique marker, made their visualization difficult. Since LT-HSCs are quiescent, or slow cycling, they are able to retain labeled nucleotides for a relatively long period and can be identified as BrdU long-term retaining (LTR) cells. Thus, mice were fed with water containing BrdU (0.8 mg/ml) for 10 days, during which time 40% of LT-HSCs would divide at least once (all the LT-HSCs divide at least once within a one-month time frame). Seventy days after BrdU labeling, bone and bone marrow sections were stained with anti-BrdU antibody. 76% of the BrdU-LTR cells were located within the bone marrow cavity, while 24% were attached to the bone surface. The majority of the BrdU-LTR cells that were attached to the bone surface were located in cancellous/trabecular bone area (including epiphysis and metaphysis), shown in FIG. 6b, whereas, the rest were dispersed along the endosteal surface of long bone, as shown in FIG. 6d. This was consistent with previous observations in which HSCs were found to be close to the endosteal surface of the long bone, or homing to the bone surface of epiphysis. Importantly, a significantly increased number of BrdU-LTR cells were found in the ectopically formed TBLA, as shown in FIG. 6c, as compared to the long bone region, shown in FIG. 6d. These BrdU-LTR cells were comprised of different types of cells including Lin⁻CD45⁺ (enriched with LT-HSCs), Lin⁺CD45⁺ (enriched with memory T or B cells that are, in most cases, in an arrested state), and Lin⁻CD45⁻ (enriched with mesenchymal stem cells).

Utilizing the staining procedure diagrammed in FIG. 6i, these populations of BrdU-LTR cells were distinguished. The majority of BrdU-LTR cells attached to the TB surface could be co-stained with Sca-1 (74%), c-Kit (72%), or a pan-hematopoietic marker, CD45 (92%). BrdU-LTR Lin⁺CD45⁺ were enriched with memory T or B cells, BrdU-LTR Lin⁻CD45⁺ were enriched with LT-HSCs, and BrdU-LTR Lin⁻CD45⁺ were enriched with mesenchymal stem cells. BrdU-LTR cells were co-stained with a cocktail of hematopoietic lineage positive markers (B220, CD19, lgM, CD2, CD4, CD8 and Gr1), and also with a pan-hematopoietic marker CD45. It was observed that 1) within the BrdU-LTR cells in bone marrow, up to 93% BrdU-LTR cells were Lin⁺CD45⁺, showing that the majority of BrdU-LTR cells within bone marrow are Lin⁺ (mainly T or B cells revealed by individual antibody staining, not shown), 4% were Lin⁻CD45⁻, and 3% were Lin⁻CD45⁺;2) within the BrdU-LTR cells attached to the bone surface, 18% were Lin⁺CD45⁺, 75% were Lin⁻CD45⁺ cells, and 7% were Lin⁻CD45⁻. The percentages listed in parentheses are based on the comparison of the positive cells with the counted cells. All these are on based on a count of 18 slides, each slides have an average 364/18=20 BrdU-LTR cells. There are 10⁵ bone marrow cells were estimated in each section (slide).

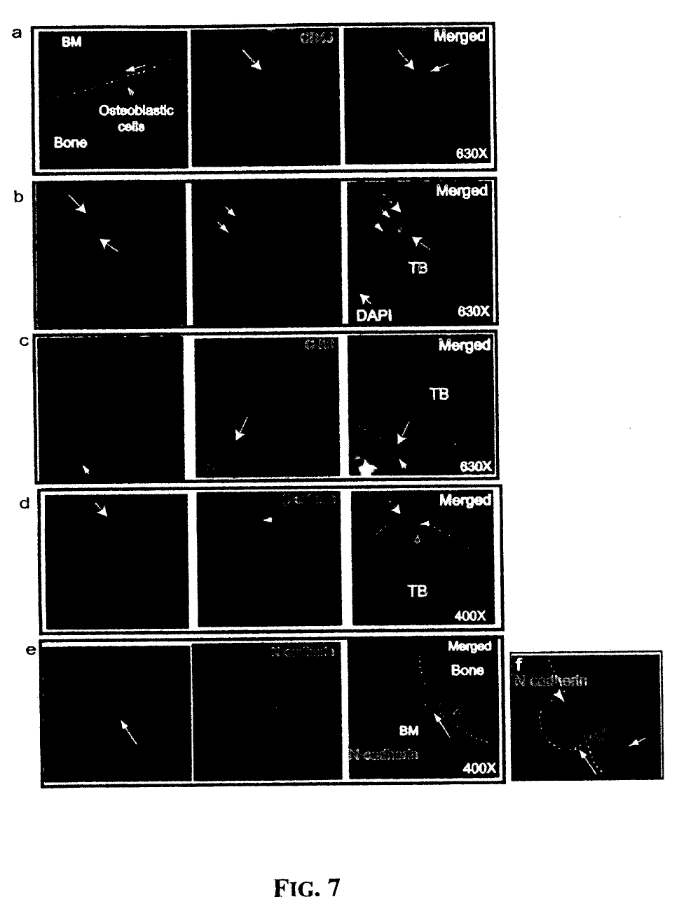

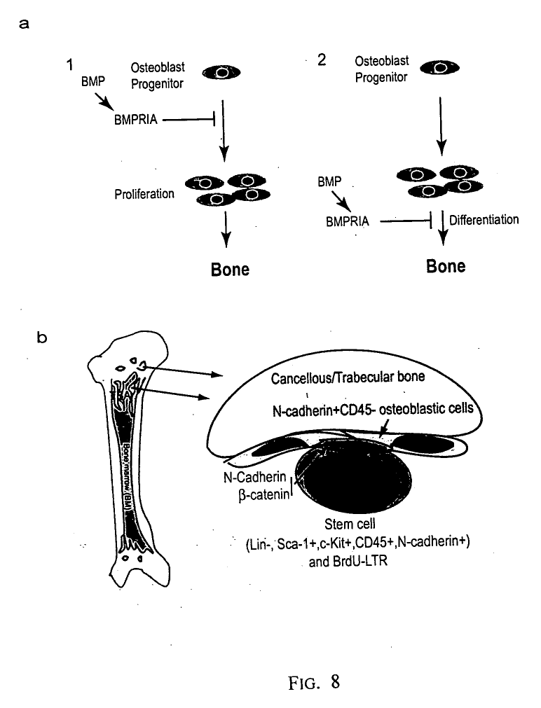

The estimated LT-HSCs was calculated as follow: 20×=[(3%×76%)+(75%×24%)]/40%=1-BrdU cells in 10⁵ bone marrow or 0.01%. 40% is the BrdU labeling efficiency. The frequency of the enriched LT-HSCs (BrdU-LTR Lin⁻CD45⁺ cells) was estimated to be 0.01%, and was close to the frequency of LT-HSCs (0.007%) based upon functional studies. These cells were mainly located on the bone surface, particularly the surface of the cancellous/trabecular bone, and were also co-stained with other HSC markers, Sca-1 and c-Kit, shown in FIGS. 7b and 7c. These observations support the conclusion that the ectopically formed TBLA is responsible for the increase in the number of HSCs.

### Example 9

The increase in the number of osteoblasts on the surface of ectopically formed TBLA correlates with the increase in the number of HSCs. It was observed that there was a physical relationship between the Lin⁻CD45⁺BrdU-LTR cells and the cells with an early osteoblastic character (the mononuclear spindle-shaped cells lining the bone surface), as shown in FIGS. 7b-7d. However, a biological marker was required to confirm this observation. Since N-cadherin is expressed in both early and late osteoblastic cells, it can be used as an osteoblastic cell marker. This marker stains two osteoblastic cell types: a small subset of spindle-shaped osteoblasts (osteoblastic lining cells), shown in FIGS. 6e-6g and the majority of the larger, oval-shaped matrix-forming osteoblasts, as shown in FIG. 6e. Histological analysis of the distribution of the spindle-shaped N-cadherin⁺ osteoblastic cells revealed that these cells were enriched on the surface of cancellous/trabecular bone, including the vesicle area in epiphysis, and sporadically dispersed along on the endosteal surface of long bone, shown in FIGS. 6e-6g. This distribution pattern was similar to that of Lin⁻BrdU-LTR cells, as shown in FIGS. 6b-6d. Indeed, the Lin⁻BrdU-LTR cells were found to be attached only to the spindle-shaped N-cadherin⁺ osteoblastic cells lining the bone surface, as shown in FIG. 7e. Furthermore, the number of spindle-shaped N-cadherin⁺ cells were counted, and the result showed that an increase in the number of these cells (2.3-fold), shown in FIG. 6h, correlated with the increase in the LT-HSC number (2.2-fold), shown in FIG. 4a, in the Bmpr1a mutant mice. Four sections derived from four different mice corresponding to either Wt or Bmpr1a mutant mice were counted by three persons in a blind procedure. An average of total number of the spindle-shaped N-cadherin⁺ osteoblastic cells per section is shown. Taken together, these observations indicate that the spindle-shaped N-cadherin⁺ osteoblast cells play an important role in supporting LT-HSCs.

### Example 10

Since the niche functions include adhesive interaction between stem cells and the niche, it was analyzed whether the spindle-shaped N-cadherin⁺CD45⁻ cells, shown in FIG. 7a, are distinguishable from hematopoietic cells and provide an adhesive attachment for HSCs. In Drosophila, two important junction-related adherens molecules, DE-cadherin and β-catenin, have been shown to be essential for the maintenance of ovarian somatic stem cells, as evidenced by an observation of a loss of DE-cadherin leading to a loss of somatic stem cells. It was analyzed to determine whether these adherens-junction-related molecules were also present between LT-HSCs and the spindle-shaped N-cadherin⁺CD45⁻ cells. Using immunohistochemical staining, it was found that E-cadherin was expressed in neither osteoblasts nor LT-HSCs, but was expressed in a majority of the bone marrow cells. However, N-cadherin was asymmetrically localized to the cell surface of BrdU-LTR cells adjacent to the osteoblastic cells, as shown in FIGS. 7e and 7f. Using flow cytometric assay, it was confirmed that expression of N-cadherin is present in a sub-population (10%) of murine adult HSCs (Lin⁻Sca-1⁺c-Kit⁺). N-cadherin-low HSCs were analyzed with Sca-1-PE and c-Kit-APC, together with anti-N-cad antibody (clone YS, Japan). Subsequently, gating on a Lin⁻ population was used, as well as in human CD34⁺ hematopoietic stem cells, as previously reported. This is shown in FIG. 13. In addition, β-catenin interacts with and forms an adherens complex with N-cadherin, and is also found to be asymmetrically localized between the osteoblastic cells and BrdU-LTR cells, shown in FIG. 7d. This specific asymmetric localization of N-cadherin and β-catenin between the spindle-shaped N-cadherin⁺CD45⁻ cells and LT-HSCs provides a possible mechanism by which the osteoblastic cells may function as a niche for HSCs.

In summary, as illustrated in FIG. 8b, the data supports the following conclusions. The spindle-shaped N-cadherin⁺CD45⁻ osteoblastic cells, which are mainly located on the surface of cancellous/trabecular bone, are a key component of the resident niche for HSCs. Asymmetrical localization of N-cadherin and β-catenin between spindle-shaped N-cadherin⁺CD45⁻ cells and HSC plays an important role in the maintenance of stem cells. In bone and bone marrow, the cancellous/trabecular bone area is the primary site for HSCs. The spindle-shaped N-cadherin⁺CD45⁻ osteoblastic cells located on the bone surface are a key component of the niche in supporting LT-HSCs (Lin⁻Sca-1⁺c-Kit⁺CD45⁺N-cad⁺), as shown in FIG. 8b. This shows that the number of the spindle-shaped N-cadherin⁺CD45⁻ osteoblastic cells is negatively controlled by the BMP signal through Bmpr1a.

Bmpr1a may affect either proliferation or differentiation of osteoblast progenitors, as shown in FIG. 8a. These osteoblast activities may be influenced through the Bmpr1a gene and/or receptor. In one model, Bmpr1a signal inhibits osteoblastic progenitor cell proliferation. Over proliferation of osteoblasts resulted from blockage of Bmpr1a. In a second model, the Bmpr1a signal inhibits osteoblastic progenitor cell differentiation. Over-production of the osteoblastic lineage resulted from blockage of the Bmpr1a-mediated signal. Alternatively, inactivation of Bmpr1a may also make osteoblasts less sensitive to apoptotic signal as BMP signal can induce osteoblast apoptosis. Either of these mechanisms can result in an increase in the number osteoblasts and the related bone hyperplasia. One of the functions of the spindle-shaped N-cadherin⁺CD45⁻ cells in supporting HSCs may be through a specific adhesive interaction between N-cadherin and β-catenin. β-catenin may play a role as a switch from an N-cadherin-associated form to an activated form required for the self-renewal of HSCs. Strikingly, in coll-PPR transgenic mice, trabecular bone volume also correlates with the HSC numbers; and the osteoblastic lineage is defined as a key participant in regulation of HSC numbers.

### Example 11

The Flow Cytometric Assay and CFU culture methods were as follows. Isolation and preparation of bone marrow, thymus, spleen, and peripheral blood cells, and the method for subsequent flow cytometric assays have been described. Briefly, bone marrow cells were flushed from femurs and tibias into PBS with 1% fetal bovine serum (FBS). Peripheral blood cells were collected in the presence of anticoagulant citrate dextrose (ACD). Red blood cells were lysed from specimens using an ammonium chloride solution.

For HSC analysis, mononuclear bone marrow cells were stained with FITC-lineage markers (CD4, CD8, CD3, B220, IgM, Mac-1, Gr-1, Ter-119, for Lin+), APC-c-Kit, and PE-Sca-1 for HSCs. For lineage analyses, see FIGS. 9 and 10. CFU assay was performed following the manufacturer's recommendations (StemCell Technology, Canada).

### Example 12

The Competitive Repopulation Unit (CRU) assay method was as follows. CRU assay was performed following the reported procedure. Briefly, a series of diluted bone marrow mononuclear cells (1×10⁴, 3×10⁴, 1×10⁵, 3×10⁵) from either Bmpr1a mutant or Wt control were transplanted into several groups of sub-lethally (500 Rads) irradiated Ly5.1 recipient female mice. Three months after transplantation, peripheral blood was analyzed using myeloid and lymphoid lineage markers: Mac-1, Gr-1, B220, CD3, and donor marker Ly5.2. An engraftment rate>1% (the basal level was defined by transplantation of bone marrow derived from Ly5.1 recipient mice) was scored as positive. The data shown in FIG. 4c are based on two experiments. The results were combined from these two independent experiments for each group with different diluted bone marrow cells (1×10⁴ group: Wt 0/5 and Mutant 0/5, 3×10⁴ group: Wt 0/7 and Mutant 1/7, 1×10⁵ group: Wt 3/13 and Mutant 8/16, 3×10⁵ group: Wt 2/6 and Mutant 3/5) and using L-Calc software (StemCell Technology) to determine the frequency of CRU in Wt and Bmpr1a mutant mice.

### Example 13

The Immunohistochemical Staining and BrdU-LTR assay method was as follows. The procedure for BrdU pulse labeling and LTR and the subsequent detection has been reported. Fresh bone specimens were embedded in OCT medium (Sakura) and snap-frozen in cooled (−48° C.) isopentane. Frozen sections of undecalcified bone were obtained using the CryoJane tape transfer system (Instrumedics) and a tungsten carbide knife. The 5-micron thick sections were air-dried overnight and stored in a −80° C. freezer until needed. On the day of use, they were thawed at room temperature for 10 minutes, and immunostained following the protocol below, except for omission of the epitope unmasking step. For paraffin-embedded sections, mouse bone specimens were collected and fixed overnight in zinc formalin (Richard-Allan Scientific) at room temperature, decalcified in Formic Acid Bone Decalcifier (Immunocal) at room temperature for 48 hours, dehydrated, embedded in wax, and sectioned at 4 μm. After deparaffinization following standard procedures, epitope unmasking was accomplished using 10 mM citrate buffer (pH 6.0) in a 70° C. water bath for 3-4 hours, then cooled for 10 to 20 minutes.

The sections were rinsed three times using double-distilled water, followed by 5 minutes of 3% hydrogen peroxide treatment at room temperature for blocking the endogenous peroxidase. Endogenous biotin was blocked when applicable using the Avidin/Biotin block kit (Vector Laboratories, Inc.). Non-specific antibody binding was blocked using a combination of 2% normal mouse serum and 10% normal goat serum in PBS for 30 minutes prior to application of primary antibody. BrdU co-staining with CD45, c-Kit, N-cadherin, β-catenin, and Lin+ markers was accomplished using a cocktail of purified primary antibody diluted with anti-BrdU-Biotinylated antibody (Zymed—ready-to-use) and incubated overnight at 4° C. They were subsequently rinsed three times in PBS/Tween and then incubated in a cocktail of Streptavidin and species-specific secondary antibody for 1 hour at room temperature, then rinsed 3-5 times with DI water. The sections were mounted with DAPI Blue Fluorescent mounting medium (Innogenex) and then viewed using the appropriate filters.

The following antibodies were used for single and double-immunostaining: Anti-Bmpr1a antibody (1:500; a gift from Dr. P. Dijke, The Netherlands Cancer Institute, Amsterdam), Rat anti-mouse CD45 (1:20, BD), Rat anti-mouse CD2/CD4/CD5/CD8/CD19/B220/IgM/Gr1 cocktail (1:20 for each antibody, BD), Rabbit anti-C-kit (1:50, Santa Cruz Biotechnology), Rabbit anti-β-catenin (1:200, Sigma), Rabbit anti-E-cadherin (1:200, Zymed), Rabbit anti-human N-cadherin (1:40, IBL), and Rat anti-mouse Sca-1 (D7, Pharmingen, 1:50) for frozen section. The secondary reagents used in immuno-fluorescence staining were all from Molecular Probes: Goat anti-rat Alexa 488 (1:200), Goat anti-Rabbit Alexa 546 (1:100), Goat anti-rat Alexa 546 (1:100), Streptavidin (SA)-Alexa 647 (1:200), Streptavidin (SA)-Alexa 488 (1:200), and Goat anti-rat Alexa 594 (1:200). The parenthetical ratios stated are weight ratios.

### Example 14

The N-Cadherin-Positive Cell Count Method was as follows. For quantitative analysis of N-cadherin-positive cells, the sections were developed with AEC after being incubated with Rabbit anti-N-cadherin antibody for 1 hour and HRP-Goat anti-rabbit second antibody for 1 hour. Three persons, in separate, blind studies, counted the spindle-shaped N-cadherin positive cells throughout the sections.

### Example 15

The X-Ray Image Method was as follows. High resolution x-rays (Faxitron MX-20, Buffalo Grove, Ill.) of bone were performed at the University of Missouri-Kansas City School of Dentistry.

### Example 16

The Inducible Cre Expression Method was as follows. PolyI:C (250 μg/mouse) was injected intra-peritoneally on the days indicated in the text.

### Example 17

This Example pertains to the in vitro co-cultivation of HSCs with the N-cad⁺CD45⁻ osteoblast cells, wherein the feeder layer is derived from the osteoblast cells. Briefly, murine or human HSCs can be cultivated with osteoblasts as the feeder layer.

The osteoblasts can be cultivated in RMPI-1640 medium (GIBCO/BRL, Rockville, Md.) or MEM-α (GIBCO/BRL) supplemented with 10% (v/v) horse serum (GIBCO/BRL) (hereinafter, “RMPI-Complete” and MEM-α-Complete” medium). The osteoblast cells are grown at 37° C. in T-25, T-75, and T-150 tissue culture flasks in a 5% CO₂ humidified environment. Just prior to reaching confluence, the osteoblast cells are trypsinized in MEM, then placed in 12-well plates in MEM-α-Complete medium and reincubated (hereinafter “conventional microplates”). Alternatively, the cell culture insert system (Becton Dickinson Labware; Franklin Lakes, N.J.) can be used. The osteoblast cells are cultured on the reverse side of the track-etched membrane of the insert for 12-well microplates in MEM-α-Complete medium (hereinafter “membrane microplates”). After the osteoblast cells are grown to confluence on the conventional and membrane microplates, microplates containing the osteoblast feeder layers are irradiated with 15 Gy radiation dose (1 Gy=1 joule/kilogram) using a ¹³⁷Cs-γ-irradiation source.

Isolated murine and human HSCs can be obtained as previously described. Serum-free liquid co-cultivation of the murine HSC and the osteoblasts on conventional and membrane microplates can be performed as follows: Serial two-fold aliquots of cells ranging from 1×10³ to 1×10⁶ cells per well of either HSC or osteoblasts or both are placed into each well of the 12-well microplate. The serum-free medium used is StemPro™_{—}34SFM (GIBCO/BRL), supplemented with StemPro™-34 Nutrient Supplement (GIBCO/BRL), 2 mM L-glutamine (GIBCO/BRL), and penicillin-streptomycin (GIBCO/BRL).

For conventional microplates, isolated HSCs are seeded directly into wells containing osteoblast feeder cell layers. In contrast, for membrane microplates, isolated HSCs are seeded on the upper side of the membrane of the insert. Osteoblast cells, located on the lower side of the membrane, extrude their microvilli into the upper side of the membrane through the etched 0.45 μm pores (pore density 1.0×10⁸/cm²). While osteoblast cells contact HSCs during HSC clonal growth and expansion, expanded HSC cells can readily be removed from cultures without contamination with the osteoblasts. On days 5, 7, 10 and 14 of culture at 37° C., aliquots of cultured cells can be harvested, enumerated, analyzed, and characterized by flow cytometry, immunological surface marker, histological staining, and mouse transplantation methods.

In summary, this Example describes the in vitro cultivation of murine and human HSCs in the presence of osteoblasts on feeder cells in 12-well membrane and conventional microplates. Expanded HSCs can be harvested, counted, analyzed, and characterized by various described methodologies.

### Example 18

This Example pertains to the in vitro co-cultivation of HSCs with the N-cad⁺CD45⁻ osteoblast cells isolated in Example 14, on an osteoblast feeder layer attached to microplate wells using poly-L-lysine. Briefly, murine and human HSCs can be cultivated with osteoblast feeder cell layer.

N-cad⁺CD45⁻ osteoblast cell feeder layers can be adhered to the bottoms of 12-well microplates utilizing 0.01 to 0.1% poly-L-lysine (PLL) (Sigma-Aldrich MDL# MFCD00165424, St. Louis, Mo.; where 0.01%=0.01 g/100 ml)), with a molecular weight (MW) of 70,000-150,000 daltons. In this method, 0.01 to 0.1% poly-L-lysine is dissolved in distilled water to give a final concentration of 0.01 to 0.1% (g/100 ml). For example, Sigma's recommended concentration is to use 0.5 ml of a 0.1 mg/ml solution to coat a surface area of 25 cm². PLL coating of wells normally occurs for approximately 4 hrs at 4° C. However, other incubation temperatures and times may also be effective. The solution is then aspirated, and the wells are rinsed twice with distilled water. Osteoblast cells can be suspended in PBS and placed into PLL-coated microwells for 4 hrs at 4° C. During this incubation period, osteoblasts can adhere to the PLL-coated bottoms of the microwells. After incubation, wells containing osteoblast feeder layers are blocked with an excess volume of 5% (wt./vol.) mouse serum albumin (MSA), mouse serum, bovine serum albumin (BSA), or bovine serum solution in PBS for 4 hrs at 4° C. Feeder layers are then washed five times with MEM-α or phosphate-buffered saline (PBS) to remove the blocking protein. After the osteoblasts are coated confluently on the wells of microplates, some, but not all, of the microplates containing the osteoblast feeder layers are irradiated with 15 Gy radiation dose (1 Gy=1 joule/kilogram) using a ¹³⁷Cs-γ-irradiation source.

Isolated murine and human HSCs can be obtained as previously described. Isolated murine or human HSCs are seeded directly into wells containing either unirradiated or irradiated osteoblast feeder cell layers. Serum-free liquid co-cultivation of the isolated HSCs and the osteoblast feeder layer on conventional microplates can be performed as follows: Serial two-fold aliquots of isolated HSCs ranging from 1×10³ to 1×10⁶ cells per well are placed into each well of the 12-well osteoblast-coated microplates. The serum-free medium used is StemPro™_{—}34SFM (GIBCO/BRL), supplemented with StemPro™-34 Nutrient Supplement (GIBCO/BRL), 2 mM L-glutamine (GIBCO/BRL), and penicillin-streptomycin. On days 5, 7, 10 and 14 of culture at 37° C., aliquots of cultured cells can be harvested, enumerated, analyzed, and characterized by flow cytometry, immunological surface marker, histological staining, and mouse transplantation methods. HSC proliferation and differentiation is anticipated to occur upon exposure of HSCs to unirradiated osteoblast feeder layers. Reduced HSC proliferation and differentiation may occur with exposure of HSCs to irradiated osteoblast feeder layers.

In summary, this Example describes the in vitro cultivation of murine and human HSCs in the presence of osteoblast murine feeder cells bound to wells of 12-well microplates using poly-L-lysine. Expanded HSCs can be harvested, counted, analyzed, and characterized by various described methodologies.

### Example 19

This Example pertains to the in vitro co-cultivation of HSCs with the N-cad⁺CD45⁻ osteoblast cells isolated in Example 14, in serum free conditions using the Matrigel Matrix (Becton Dickinson, Mountain View, Calif.) culture system. The Becton Dickinson (BD) Matrigel Matrix is a solubilized basement membrane extract obtained from the Engelbreth-Holm-Swarm EHS mouse sarcoma, a tumor rich in extracellular matrix (ECM) proteins. Its major component is laminin, followed by collagen IV, heparan sulfate proteoglycans, entactin and nidogen.

Matrigel Basement Membrane Matrix (Becton Dickinson Product #356234) can be used in either a thin gel method or thick gel method for coating wells of plates and growing cells in vitro. Alternatively, growth factor-reduced Matrigel (#356231) can be used. To prepare Matrigel aliquots, slowly thaw Matrigel at 4° C. overnight to avoid gel formation. The serum-free medium (Growth Medium) used is StemPro™_{—}34SFM (GIBCO/BRL), supplemented with StemPro™-34 Nutrient Supplement (GIBCO/BRL), 2 mM L-glutamine (GIBCO/BRL), and penicillin-streptomycin.

Add 10 ml of cold Growth Medium to a bottle containing 10 ml Matrigel. Mix by pipetting on ice. Aliquot 1-2 ml into each pre-chilled tube, and store aliquots at −20° C. Slowly thaw Matrigel aliquots at 4° C. for at lest 2 hr to avoid formation of a gel. Dilute Matrigel aliquots 1:15 in cold Growth Medium for a final dilution of 1:30.

Add 1 ml of Matrigel solution to coat each well of a 6-well plate (Falcon #3046). Incubate the plates 1-2 hr at RT, or at least overnight at 4° C. The Matrigel solution is removed immediately before use.

In the thin gel method, osteoblasts, isolated according to Example 14, can be suspended in Matrigel Basement Membrane Matrix with cooled pipettes such that there is 50 microliters per square centimeter of growth surface. Plates containing osteoblast-coated wells are incubated for 30 min. at 37° C. In the thick gel method, 150-200 microliters per square centimeter of growth surface is used. Serial two-fold aliquots of osteoblast cells ranging from 1×10⁴ to 1×10⁷ cells per well are placed into each well of the 6-well plate.

HSCs with the N-cad⁺CD45⁻ osteoblast cells isolated in Example 14 and suspended in Growth Medium can be seeded onto the surfaces of each well of the Matrigel-coated plates. The final volume is 4 ml per well. Serial two-fold aliquots of cells ranging from 1×10⁴ to 1×10⁷ cells per well of HSC are placed into each well of the 6-well plate. On days 5, 7, 10 and 14 of culture at 37° C., aliquots of cultured cells can be harvested, enumerated, analyzed, and characterized by flow cytometry, immunological surface marker, histological staining, and mouse transplantation methods.

In summary, this Example describes the in vitro cultivation of murine HSCs in the presence of osteoblasts in a Matrigel feeder-free system in 6-well plates.

Thus, there has been shown and described an identification of the hematopoietic stem cell niche and control of the niche size, which fulfills all the objects and advantages sought therefor. It is apparent to those skilled in the art, however, that many changes, variations, modifications, and other uses and applications to the identification of the hematopoietic stem cell niche and control of the niche size are possible, and also such changes, variations, modifications, and other uses and applications which do not depart from the spirit and scope of the invention are deemed to be covered by the invention, which is limited only by the claims which follow.

### REFERENCE LIST

1. Spangrude, G. H., Heimfeld, S., Weissman, I. L. Purification and characterization of mouse hematopoietic stem cells. Science 241, 58-62 (1988).

2. Miller, C. L. & Eaves, C. J. Expansion in vitro of adult murine hematopoietic stem cells with transplantable lympho-myeloid reconstituting ability. Proc Natl Acad Sci U S A 94, 13648-53. (1997).

3. Verfaillie, C. M. et al. The hematopoietic microenvironment: stromal extracellular matrix components as growth regulators for human hematopoietic progenitors. Hematol 4, 321-333 (1999).

4. Park, I., H E, Y., Lin, F., Laerum, O. D., Tian, Q., Bumgarner, R., Klug, C., Li, K., Kuhr, C., Doyle, M., Xie, T., Schummer, M., Sun, Y., Goldsmith, A., Clarke, M. F., Weissman, I. L., Hood, L. and Li, L. Differential gene expression profiling of adult murine hematopoietic stem cells. Blood 99, 488-98 (2002).

5. Akashi, K. et al. Transcriptional accessibility for genes of multiple tissues and hematopoietic lineages is hierarchically controlled during early hematopoiesis. Blood 101, 383-9 (2003).

6. Maeno, M. et al. The role of BMP-4 and GATA-2 in the induction and differentiation of hematopoietic mesoderm in Xenopus laevis. Blood 88, 1965-72 (1996).

7. Davidson, A. J. & Zon, L. I. Turning mesoderm into blood: the formation of hematopoietic stem cells during embryogenesis. Curr Top Dev Biol 50, 45-60 (2000).

8. Bhatia, M. et al. Bone morphogenetic proteins regulate the developmental program of human hematopoietic stem cells. J Exp Med 189, 113-48 (1999).

9. Yi, S. E., Daluiski, A., Pederson, R., Rosen, V. & Lyons, K. M. The type I BMP receptor Bmpr1b is required for chondrogenesis in the mouse limb. Development 127, 621-30 (2000).

10. Mishina, Y., Suzuki, A., Ueno, N. & Behringer, R. R. Bmpr encodes a type I bone morphogenetic protein receptor that is essential for gastrulation during mouse embryogenesis. Genes Dev 9, 3027-37 (1995).

11. Mishina, Y., Hanks, M. C, Miura, S., Tallquist, M. D., & Behringer, R. R. Generation of Bmpr/Alk3 conditional knockout mice. Genesis 32 69-72 (2002).

12. Kuhn, R., Schwenk, F., Aguet, M. & Rajewsky, K. Inducible gene targeting in mice. Science 269, 1427-9 (1995).

13. Novak, A., Guo, C., Yang, W., Nagy, A. & Lobe, C. G. Z/EG, a double reporter mouse line that expresses enhanced green fluorescent protein upon Cre-mediated excision. Genesis 28, 147-55 (2000).

14. Morrison, S. J., Weissman, I. L. The long-term repopulating subset of hematopoietic stem cells is deterministic and isolatable by phenotype. Immunity, 661-672 (1994).

15. Thorsteinsdottir, U., Sauvageau, G. & Humphries, R. K. Enhanced in vivo regenerative potential of HOXB4-transduced hematopoietic stem cells with regulation of their pool size. Blood 94, 2605-12 (1999).

16. Kondo, M., Weissman, I. L. & Akashi, K. Identification of clonogenic common lymphoid progenitors in mouse bone marrow. Cell 91, 661-72 (1997).

17. Akashi, K., Traver, D., Miyamoto, T. & Weissman, I. L. A clonogenic common myeloid progenitor that gives rise to all myeloid lineages. Nature 404, 193-7 (2000).

18. Simmons, P., Gronthos, S., Zannettino, A C. In Hematopoiesis: A developmental approach (ed. Zon, L. I.) 718-726 (Oxford University Press, New York, 2001).

19. Charbord, P. The hematopoietic stem cell and the stromal microenvironment. Therapie 56, 383-4 (2001).

20. Taichman, R. S., Reilly, M. J. & Emerson, S. G. The hematopoietic microenvironment: Osteoblasts and the hematopoietic microenvironment. Hematol 4, 4210426 (2000).

21. El-Badri, N. S., Wang, B. Y., Cherry & Good, R. A. Osteoblasts promote engraftment of allogeneic hematopoietic stem cells. Exp Hematol 26, 1106 (1998).

22. Chen, D. et al. Differential roles for bone morphogenetic protein (BMP) receptor type IB and IA in differentiation and specification of mesenchymal precursor cells to osteoblast and adipocyte lineages. J Cell Biol 142, 295-305 (1998).

23. Taylor, G., Lehrer, M. S., Jensen, P. J., Sun, T. T. & Lavker, R. M. Involvement of follicular stem cells in forming not only the follicle but also the epidermis. Cell 102, 451-61 (2000).

24. Cheshier, S. H., Morrison, S. J., Liao, X. and Weissman, I. L. In vivo proliferation and cell cycle kinetics of long-term self-renewing hematopoietic stem cells. Proc Natl Acad Sci U S A 96, 3120-5 (1999).

25. Nilsson, S. K., Johnston, H. M. & Coverdale, J. A. Spatial localization of transplanted hemopoietic stem cells: inferences for the localization of stem cell niches. Blood 97, 2293-9 (2001)

26. Heissing, B. et al. Recruitment of stem and progenitor cells from the bone marrow niche requires MMP-9 mediated release of kit-ligand. Cell 109, 625-37 (2002).

27. Askenasy, N. & Farkas, D. L. Optical imaging of PKH-labeled hematopoietic cells in recipient bone marrow in vivo. Stem Cells 20, 501-513 (2002).

28. Lagasse, E., Shizuru, J A., Uchida, N., Tsukamoto, A., Weissman, I L., Toward regenerative medicine. Immunity 14, 425-436 (2001).

29. Hay, E. et al. N- and E-cadherin mediate early human calvaria osteoblast differentiation promoted by bone morphogenetic protein-2. J Cell Physiol 183, 117-28 (2000).

30. Visnjic, D. et al. Conditional ablation of the osteoblast lineage in Col2.3deltak transgenic mice. J Bone Miner Res 16, 2222-31 (2001).

31. Spradling, A., Drummond-Barbosa, D. & Kai, T. Stem cells find their niche. Nature 414, 98-104 (2001).

32. Song, X. & Xie, T. DE-cadherin-mediated cell adhesion is essential for maintaining somatic stem cells in the Drosophila ovary. Proc Natl Acad Sci U S A 99, 14813-8 (2002).

33. Puch, S. et al. N-cadherin is developmentally regulated and functionally involved in early hematopoietic cell differentiation. J Cell Sci 114, 1567-77 (2001).

34. Hay, E., Lemonnier, J., Fromigue, 0. & Marie, P. J. Bone morphogenetic protein-2 promotes osteoblast apoptosis through a Smad-independent, protein kinase C-dependent signaling pathway. J Biol Chem 276, 29028-36 (2001).

35. Reya, T. et al. A role for Wnt signalling in self-renewal of haematopoietic stem cells. Nature 423, 409-14 (2003).

36. Schofield, R. The relationship between the spleen colony-forming cell and the hamatopopietic stem cell. A hypothesis. Blood Cells 4, 7-25 (1978).

37. Dexter, T. M., Allen, T. D. & Lajtha, L. G. Conditions controlling the proliferation of haemopoietic stem cells in vitro. J Cell Physiol 91, 335-44 (1977).

38. Weissman, I. L. Developmental switches in the immune system. Cell 76, 207-218 (1994).

39. Watt, F. M. & Hogan, B. L. Out of Eden: stem cells and their niches. Science 287, 1427-30 (2000).

40. Lin, H. The stem-cell niche theory: lessons from flies. Nat Rev Genet 3, 931-40 (2002).

41. Xie, T. & Spradling, A. C. A niche maintaining germ line stem cells in the Drosophila ovary. Science 290, 328-30 (2000).

42. Wagers, A. J., Sherwood, R. I., Christensen, J. L. & Weissman, I. L. Little evidence for developmental plasticity of adult hematopoietic stem cells. Science 297, 2256-9 (2002).

What is claimed is:

1. A vector comprising a mutant Bmpr1a nucleic acid sequence flanked by recombination sites.

2. The vector of claim 1, wherein the vector is an inducible Cre/Flp recombinase expression vector.

3. The vector of claim 1, wherein the recombination sites are LoxP sites.

4. A host organism transfected with the vector of claim 1.

5. Progeny from the host organism of claim 4, wherein the host of claim 4 is crossed with a host transfected with a Cre recombinase vector.

6. A vector comprising sequences homologous to the mutant Bmpr1a nucleic acid sequence of claim 1.

7. A mouse transfected with vector of claim 1.

8. A host organism comprising a homologous Bmpr1a recombination sequence and a regulatory element.

9. The organism of claim 8, wherein the organism comprises an inducible Cre/lox system, whereby mutant Bmpr1a is flanked by lox sites.

10. The organism of claim 8, wherein the regulatory element is Cre.

11. A Mx1-Cre⁺ Bmpr1a^{fx/fx} mouse.

12. A knockout mouse, wherein a Bmpr1a gene has been substantially eliminated.

13. A knockout organism, wherein a Bmpr1a gene has been substantially eliminated.

14. An antibody to a Bmpr1a polypeptide.

15. A Fab fragment from the antibody of claim 14.

16. An antibody to a Bmpr1a polypeptide, wherein the antibody increases HSC population in vivo.

17. A method for increasing HSC in vivo comprising:
(a) forming a mutant Bmpr1a recombination vector;
(b) transfecting a host with the vector; and,
(c) inducing recombination in the host, wherein a wt Bmpr1a gene is knocked-out.

18. The method of claim 17, wherein the vector is delivered to embryonic cells.

19. The method of claim 18, wherein delivery of the vector is accomplished via transfection.

20. The method of claim 17, wherein the vector comprises a mutant Bmpr1a nucleic acid sequence flanked by recombination sites.

21. The method of claim 17, wherein the host is a mouse.

22. The method of claim 17, wherein the method comprises transfecting a second host with a Cre recombination vector and crossing the Cre host with the Bmpr1a host to form progeny.

23. The method of claim 22, wherein the Bmpr1a gene is knocked-out in the progeny.

24. A method for increasing HSC in vivo, comprising delivering a mutant Bmpr1a, wherein delivery is selected from the group consisting of transfection electroporation, and microinjection.

25. A method for increasing HSC in vivo comprising:
(a) forming Bmpr1a recombination vector, wherein the vector comprises a mutant Bmpr1a nucleic acid sequence flanked by recombination sites;
(b) transfecting a host with the vector, wherein the vector is delivered to embryonic cells;
(c) transfecting a second host with a Cre recombination vector;
(d) crossing the Cre host with the Bmpr1a host to form a progeny host; and,
(e) inducing recombination in the progeny host, wherein the Bmpr1a gene is knocked-out.

26. A method for making a conditional Bmpr1a knockout mouse, comprising:
(a) forming an inducible recombinase vector having a mutant Bmpr1a sequence;
(b) transfecting the vector into embryogenic stem cells;
(c) transplanting the stem cells into a female mouse;
(d) identifying progeny mice which include a knock-in of the vector in the progeny genome;
(e) forming a recombination vector;
(f) transfecting the vector into embryogenic stem cells;
(g) transplanting the recombination stem cells into a female mouse; and,
(h) crossing the recombination progeny with the recombinase progeny.

27. An isolated population of N-cad⁺CD45⁻ osteoblast cells.

28. The population of claim 27, wherein the population comprises medium.

29. A population of cells comprising N-cad⁺CD45⁻ osteoblast cells and HSC.

30. An in vitro population of cells comprising N-cad⁺CD45⁻ cells and HSC.

31. The population of claim 30, wherein the HSCs comprise Lin⁻Sca-1⁺c-Kit⁺CD45⁺N-cad⁺ HSCs.

32. The population of claim 30, wherein the osteoblasts are affixed to a substrate.

33. The population of claim 30, wherein the osteoblasts are irradiated.

34. An in vitro feeder layer for growing HSC, comprising:
(a) a substrate; and,
(b) N-cad⁺CD45⁻ osteoblast cells.

35. A method for isolating niche cells for use in supporting HSCs, comprising:
(a) isolating a population of bone marrow cells;
(b) mixing the cells with labeled cell surface markers, the cell surface markers are CD45 and N-cadherin; and,
(c) passing the cells through a FACS sorter to separate N-cad⁺CD45⁻ osteoblast cells from the population.

36. A method of using osteoblast cells to support HSCs in vitro, comprising:
(a) isolating a population of CD45⁺N-cad⁻ osteoblast cells; and,
(b) operably contacting HSCs with the osteoblast cells.

37. The method of claim 36, wherein the osteoblasts are placed on a substrate.

38. A method for making a conditional Bmpr1a knockout mouse, comprising:
(a) forming an inducible Bmpr1a/lox recombinase vector containing a mutant Bmpr1a sequence;
(b) placing the Bmpr1a vector in operable contact with an embryogenic stem cell;
(c) transplanting the stem cells into a female mouse;
(d) identifying progeny mice which include a knock-in of the vector in the progeny genome;
(e) forming a Cre recombinase vector;
(f) placing the Cre vector into embryogenic stem cells;
(g) identifying progeny which include a Cre knock-in; and,
(h) crossing the Cre progeny with the Bmpr1a progeny to form the conditional knock-out mouse.

39. The method of a Mx1⁻Cre⁺ Bmpr1a^{fx/fx} mouse of claim 38, wherein nucleic acid molecules homologous to the Bmpr1a sequences are selected from the group consisting of mutant, antisense, base-substituted, frame shift, deletion, and truncated genes.

40. The method of claim 38, wherein the vector is selected from the group consisting of expression, cloning, and viral vectors.

41. The method of claim 38, wherein the vector is selected from the group consisting of expression vectors, fusion vectors, gene therapy vectors, two-hybrid vectors, reverse two-hybrid vectors, sequencing vectors, and cloning vectors.

42. The method of claim 38, wherein nucleic acid molecules are homologous to the mutant Bmpr1a sequences, and are selected from the group consisting of genes, mRNA, cDNA, gDNA, tRNA, RNAi, SiRNA, oligonucleotides, polynucleotides, and nucleic acid sequence fragments.

43. A positive marker for use in isolating HSCs, comprising: N-cadherin.

44. The marker of claim 43, wherein the HSC is a human HSC.

45. The marker of claim 43, wherein a FACS sorter detects the marker and separates the HSC N-cad⁺ cells from remaining HSC cells.
